(19) United States
(12) Patent Application Publication
(10) Pub. No.: US 2022/0144842 A1
(43) Pub. Date: May 12, 2022

(54) IRAK INHIBITORS, PREPARATION METHOD AND MEDICINAL USES THEREOF

(71) Applicant: Eternity Bioscience Inc., Cranbury, NJ (US)

(72) Inventors: Hongbin Li, Newtown, CT (US); Bin Cao, Skillman, NJ (US); Jian Liu, Edison, NJ (US); Linghang Zhuang, Chalfont, PA (US); Tongyuan Li, Fort Lee, NJ (US); Suxing Liu, Edison, NJ (US)

(21) Appl. No.: 17/519,538

(22) Filed: Nov. 4, 2021

Related U.S. Application Data
(60) Provisional application No. 63/109,709, filed on Nov. 4, 2020.

(51) Int. Cl. C07D 487/04

(52) U.S. Cl. CPC C07D 487/04

(57) ABSTRACT

This application discloses compounds of formula (I) and (I′) useful as IRAK inhibitors and therapeutic agents for treatment of IRAK, especially IRAK-4, mediated disease or disorders, including autoimmune diseases, cancers, neurodegenerative disorders, viral diseases, and inflammatory disorders, hereditary disorders, and so on. The application also discloses pharmaceutical compositions containing these compounds, and synthetic methods and medical uses of the compounds.

### CROSS-REFERENCE TO RELATED APPLICATIONS

This application claims priority under 35 U.S.C. § 119(e) to U.S. Provisional Patent Application No. 63/109,709, filed on Nov. 4, 2020, the disclosure of which is incorporated herein by reference in its entirety.

### FIELD OF THE DISCLOSURE

The present disclosure relates to compounds and methods useful in the inhibition of the function of IRAK and accordingly may have a beneficial impact on the therapies of diseases including, but not limited to, autoimmune diseases, cancers, inflammatory disorders, and viral diseases.

### BACKGROUND OF THE DISCLOSURE

As a central signaling mediator, Interleukin-1 receptor-associated kinase 4 (IRAK4) play crucial roles in transducing the responses of the interleukin-1 (IL-1) receptors and Toll-like receptors (TLR). IRAK4 overactivation is linked with different types of human autoimmune and inflammatory diseases. A number of studies reported that all TLR/IL-1Rs with known function are involved in host defense mechanisms, either by the recognition of pathogens or as receptors for inflammatory cytokines. They play a vital role in both innate and adaptive immunity in mammals, and the signaling cascades mediated by these receptors are associated with various human diseases. IRAK proteins are key components in the signal transduction pathways mediated by interleukin-1 receptor (IL-IR), interleukin-18 receptor (IL-18R), and Toll-like receptors (TLRs).

IRAK family consists of four members, including IRAK-1, IRAK-2, IRAK-M, also denoted IRAK-3, and IRAK-4. Among them, IRAK1 and IRAK4 proteins are catalytically active kinases, whereas IRAK2 and IRAK3 are inactive pseudokinases (Jain et al., Front. immunol., 2014, 5(553): 1-8). IRAK-4 belongs to the serine/threonine kinase and is the best characterized member of the IRAK family. IRAK-4 is involved in signaling innate immune responses from both TLRs and IL-1 receptors. Innate immunity detects pathogens through the recognition of pathogen-associated molecular patterns (PAMPs) by TLRs. TLRs recognize conserved structures of both microbes and endogenous molecules. The cell surface TLRs recognize bacterial and fungal components, whereas TLRs recognizing viral or microbial nucleic acids are localized into intracellular membranes such as endosomes and phagosomes. Upon ligand binding, these receptors recruit the scaffolding adaptor protein myeloid differentiation primary response gene (88) (MyD88) through the Toll/interleukin-1 (IL-1) receptor (TIR) domain. MyD88 is the universal adaptor used by all TLRs except for TLR3. MyD88 couples to pathways that lead to the activation of transcription factors such as NF-κB (nuclear factor-κB), IRF1 (IFN-regulatory factor 1), IRF5 and IRF7 (Luke A. J., et al., Nat. Rev. Immunol. 2007, 7: 353-364). MyD88 utilizes the death domain to recruit IRAK4, thereby activating its kinase function to phosphorylate IRAK1 and IRAK2 within the myddosome complex. Phosphorylation at multiple sites allows IRAK to dissociate from the myddosome complex and activate the downstream proteins such as TNF receptor-associated factor 6 (TRAF-6), which finally can initiate downstream activation of NF-kB and MAPK signaling pathways, leading to the induction of pro-inflammatory cytokines and chemokines, such as TNF-a, IL-6, and IL-8.

Mice homozygous for disruptions in this gene display an essentially normal phenotype, and in overexpression studies, IRAK-2 and IRAK-3 can compensate for IRAK-1 loss in a mutant 293 cell line, furthermore, in vitro assay demonstrated that kinase activity of IRAK1 is not required for IL-1 downstream signaling (Knop and Martin, FEBS Letters, 1999, 448: 81-85; Thomas, J. A., et al., J. Immunol., 1999, 163:978-984; Wesche, H., et al., J Biol Chem., 1999, 274:19403-19410; Aravind, L., et al., Science, 2001, 291:1279-1284). Both IRAK-4 and MyD88 deficient humans show normal resistance to common fungi, parasites, viruses, and many bacteria, except the susceptibility to bacterial infections, particularly recurrent pyogenic bacterial infections such as Streptococcus infections (Picard, C., et al., Science, 2003, 299:2076-2079; Ku, C L., et al., J Exp Med., 2007, 204(10):2407-2022). Human germline gain-of-function MyD88 mutation which causes the constitutive activation of IRAK4 signaling led to severe arthritis (Sikora et al., 0.14JACI, 2018, 141(5):1943-1947; Picard, C., et al., Clin. Microbiol. Rev., 2011, 24(3): 490-497; Koziczak-Holbro et al., Arthritis Rheumatol., 2009, 60(6): 1661-1671). IRAK1-deficient human peripheral blood mononuclear cells (PBMCs) have normal responses to both TLR agonists and IL-1β, but the responses in IRAK4 deficient human PBMCs are totally abolished (Mina et al., PNAS, 2017, E514-E523). Mouse knock-out experiments have demonstrated an essential role for IRAK-4 in IL-IR, IL-18R and most TLR signaling. IRAK4-deficient mice also exhibit defective innate immunity and are more susceptible to bacterial infection. IRAK4 kinase dead mice are protected against antigen-induced arthritis. (Suzuki, N., et al. Nature, 2002, 416:750-756; Koziczak-Holbro et al., Arthritis Rheumatol., 2009, 60(6): 1661-1671).

IRAK4 also plays a central role in tumor growth and progression. Inhibition of IRAK4 is universally toxic towards activated B-cell diffuse large B-cell lymphoma (ABC DLBCL) but not germinal center B-cell (GCB) DLBCL cell lines, mechanistic studies demonstrated that IRAK4 inhibition potently abrogates IRAK4-mediated phosphorylation of IRAK1 and the downstream activation of NF-κB and MAPK signaling pathways resulting from the MyD88 (L265P) mutation in ABC DLBCL, leading to the suppression of secretion of the pro-inflammatory cytokines TNFα, IL-6 and IL-10 by ABC DLBCL cells. Furthermore, aberrant B cell receptor (BCR) signaling in ABC DLBCL is also engaged in tumor growth and progression, and this chronic active BCR signaling can be blocked by inhibiting Bruton's tyrosine kinase (BTK) pharmacologically. Notably, the IRAK4 inhibitors strongly synergized with BTK inhibition in killing multiple ABC DLBCL cell lines. (Ngo et al., Nature, 2011, 470(7332): 115-119; Lim et al., Blood, 2012, 120(21):625).

Toll/IL-1 receptor family members like IRAK4 are central components of host defense mechanisms in a variety of species and IRAK4-mediated signaling, downstream of TLRs and the IL-1 receptor family, bridges both innate and adaptive immunity. Both TLRs and IL-1 pathways have been linked to immune and inflammatory diseases such as chronic inflammatory diseases, including chronic arthritis, atherosclerosis, multiple sclerosis, cancers, and autoimmune disorders including rheumatoid arthritis, lupus, asthma, psoriasis, and inflammatory bowel diseases. Overall, IRAK4 plays a key role in immune and inflammatory responses and is considered as an important potential therapeutic target for autoimmune diseases, inflammatory diseases, and cancer.

Patent applications for compounds that have been published as IRAK4 inhibitors include WO2020001449A1, WO2015048281A1, WO2017004133A1, WO2017004134A1, WO2017205762A1, WO2017205766A1, WO2017205769A1, WO2016053770A1 and WO2016053771A1. However, there is still a need to develop new IRAK, especially IRAK4, inhibitors to meet the unmet medical needs.

### SUMMARY OF THE DISCLOSURE

The compounds of this disclosure inhibit the function of IRAK and accordingly may serve as therapeutic agents for the treatment of diseases including a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The present disclosure, in one aspect, provides a compound of formula (I) or (I′), or a tautomer, mesomer, racemate, enantiomer, diastereomer thereof, or mixture thereof, or a pharmaceutically acceptable salt thereof:

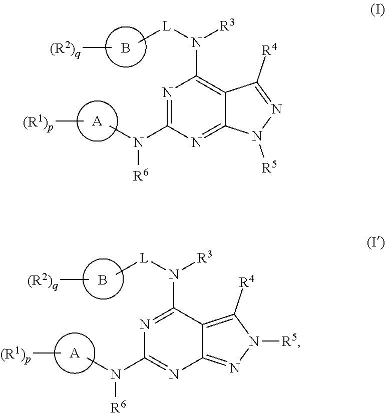

wherein;

ring A is a heteroaryl;

ring B is a cycloalkyl, heterocyclyl or heteroaryl, provided that ring B is not a piperidinyl, bridged piperidinyl or cyclopropyl;

L is a covalent bond or alkylene wherein one CH₂ group is optionally replaced with O or NH;

R¹ at each occurrence is identical or different and is independently selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{a}SO₂R^{a}, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R² at each occurrence is identical or different and is independently selected from halogen, alkyl, alkoxy, haloalkyl, haloalkoxy, hydroxyl, hydroxyalkyl, cyano, amino, nitro, —(CH₂)ᵣ—, NR^{a}R^{b}, —C(═O)R^{c}, —C(═O)OR^{a}, —OC(═O)R^{c}, —C(═O)NR^{a}R, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R³, R⁶ and R^{d} are independently selected from hydrogen, alkyl, haloalkyl, hydroxyalkyl and cycloalkyl, wherein the cycloalkyl is optionally substituted with one or more groups independently selected from halogen, alkyl, haloalkyl, alkoxy, haloalkoxy, hydroxyl, hydroxyalkyl, cyano, amino and nitro;

R⁴ is selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R⁵ is selected from hydrogen, alkyl, haloalkyl, hydroxyalkyl, C(═O)R^{c}, —C(═O)OR^{a}, —C(═O)NR^{a}R^{b}, —SO₂R^{a}, —SO₂NR^{a}R^{b}, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R^{a} and R^{b} are identical or different and at each occurrence are independently selected from hydrogen, alkyl, haloalkyl, hydroxyalkyl, cycloalkyl, heterocyclyl, aryl and heteroaryl; or R^{a} and R^{b} together with the nitrogen to which they are attached form a heterocyclyl;

R^{e} and R^{f} are identical or different and at each occurrence are independently selected from hydrogen, alkyl, haloalkyl, hydroxyalkyl, cycloalkyl, heterocyclyl, aryl, and heteroaryl; or R^{e} and R^{f} together with the nitrogen to which they are attached form a heterocyclyl:

R^{c} at each occurrence is independently selected from alkyl, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino and nitro;
p is 0, 1, 2, 3 or 4;
q is 1, 2, 3 or 4;
r is 0, 1, 2 or 3; and
s is 0, 1, 2 or 3.

In another aspect, this disclosure provides a preparation process of a compound of formula (I) or (I′), or a tautomer, mesomer, racemate, enantiomer, diastereomer, or mixture thereof, or a pharmaceutically acceptable salt thereof, the preparation process comprising the steps of.

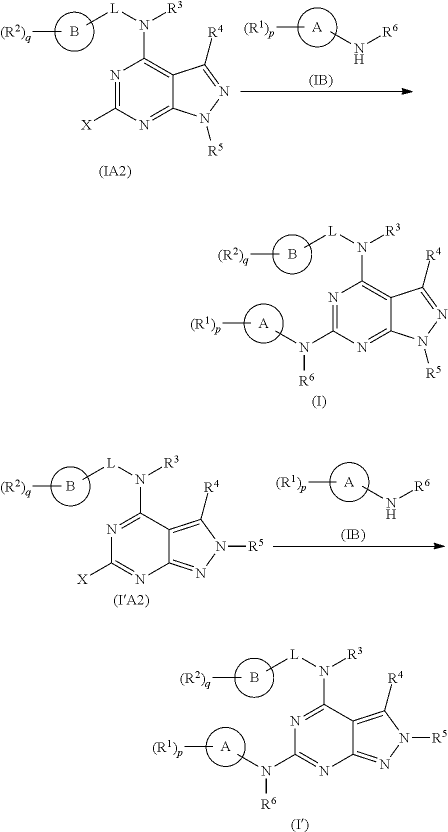

reacting a compound of formula (IA2) or (I′A2) with a compound of formula (IB) or a pharmaceutically acceptable salt thereof to obtain the compound of formula (I) or (I′), wherein:

X is halogen;

the pharmaceutically acceptable salt of the compound (IB) preferably is hydrochloride; and

ring A, ring B, L, R¹ to R⁶, p and q are each as defined in formula (I) or (I′).

In another aspect, the present disclosure provides a pharmaceutical composition comprising a compound of formula (I) or (I′), or a tautomer, mesomer, racemate, enantiomer, diastereomer, or mixture thereof, or a pharmaceutically acceptable salt thereof, and a pharmaceutically acceptable carrier.

In another aspect, the present disclosure provides a method of preventing and/or treating an autoimmune disease, a cancer, a neurodegenerative disorder, a viral disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder, comprising a step of administering to a subject in need thereof a therapeutically effective amount of a compound of formula (I) or (I′), or an isomer, pharmaceutically acceptable salt, or a pharmaceutical composition containing the compound.

In another aspect, the present disclosure also relates to use of a compound of formula (I) or (I′), or an isomer, pharmaceutically acceptable salt thereof, or a pharmaceutical composition containing the compound, in the manufacture of a medicament for treatment of a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

Other aspects or advantages of the disclosure will be better appreciated in view of the following detailed description, examples, and claims.

### DETAILED DESCRIPTION OF THE DISCLOSURE

In one aspect, the present disclosure provides a compound of formula (I) or (I′), or a tautomer, mesomer, racemate, enantiomer, diastereomer, or mixture thereof, or pharmaceutically acceptable salt thereof:

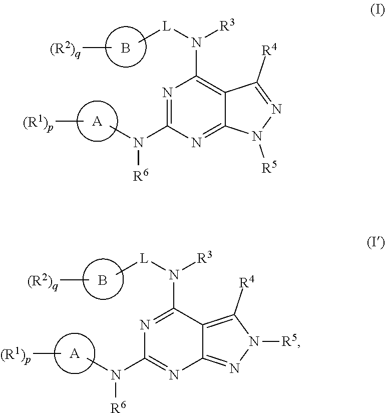

wherein:

ring A is a heteroaryl;

ring B is a cycloalkyl, heterocyclyl or heteroaryl, provided that ring B is not a piperidinyl, bridged piperidinyl or cyclopropyl;

L is a covalent bond or alkylene wherein one CH₂ group is optionally replaced with O or NH;

R¹ at each occurrence is identical or different and is independently selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R² at each occurrence is identical or different and is independently selected from halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R³, R⁶ and R^{d} are independently selected from hydrogen, alkyl, haloalkyl, hydroxyalkyl and cycloalkyl, wherein the cycloalkyl is optionally substituted with one or more groups independently selected from halogen, alkyl, haloalkyl, alkoxy, haloalkoxy, hydroxyl, hydroxyalkyl, cyano, amino and nitro;

R⁴ is selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R⁵ is selected from hydrogen, alkyl, haloalkyl, hydroxyalkyl, C(═O)R^{c}, —C(═O)OR^{a}, —C(═O)NR^{a}R^{b}, —SO₂R^{a}, —SO₂NR^{a}R^{b}, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R^{a} and R^{b} are identical or different and at each occurrence are independently selected from hydrogen, alkyl, haloalkyl, hydroxyalkyl, cycloalkyl, heterocyclyl, aryl and heteroaryl; or R^{a} and R^{b} together with the nitrogen to which they are attached form a heterocyclyl;

R^{e} and R^{f} are identical or different and at each occurrence are independently selected from hydrogen, alkyl, haloalkyl, hydroxyalkyl, cycloalkyl, heterocyclyl, aryl, and heteroaryl; or R^{e} and R^{f} together with the nitrogen to which they are attached form a heterocyclyl:

R^{c} at each occurrence is independently selected from alkyl, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino and nitro;

p is 0, 1, 2, 3 or 4;

q is 1, 2, 3 or 4;

r is 0, 1, 2 or 3; and

s is 0, 1, 2 or 3.

In some embodiments of the disclosure, in the compound of formula (I) or (I′), or a tautomer, mesomer, racemate, enantiomer, diastereomer, or mixture thereof, or a pharmaceutically acceptable salt thereof, ring B is 4-6 membered cycloalkyl; preferably is cyclohexyl.

In some embodiments of the disclosure, the compound of formula (I) or (I′), or a tautomer, mesomer, racemate, enantiomer, diastereomer, or mixture thereof, or a pharmaceutically acceptable salt thereof, is a compound of formula (II) or (II′), or a tautomer, mesomer, racemate, enantiomer, diastereomer, or mixture thereof, or a pharmaceutically acceptable salt thereof:

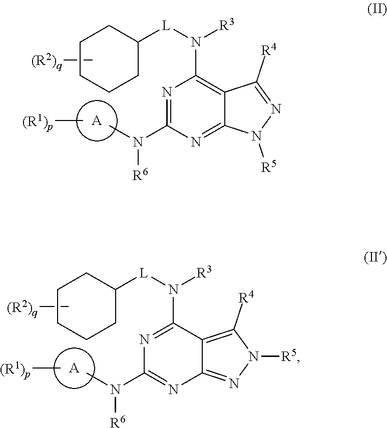

wherein ring A, L, R¹-R⁶, p and q are each as defined in formula (I) or (I′).

In some embodiments of the disclosure, the compound of formula (I) or (I′), or a tautomer, mesomer, racemate, enantiomer, diastereomer, or mixture thereof, or a pharmaceutically acceptable salt thereof, is a compound of formula (III) or (III′), or a tautomer, mesomer, racemate, enantiomer, diastereomer, or mixture thereof, or a pharmaceutically acceptable salt thereof:

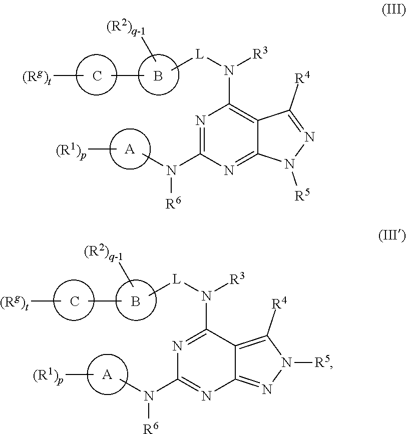

wherein:

ring C is selected from cycloalkyl, heterocyclyl, aryl and heteroaryl;

R^{g} is identical or different and at each occurrence is independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

t is 0, 1, 2 or 3;

ring A, ring B, L, R¹-R⁶, R^{f}, R^{e}, s, p and q are each as defined in formula (I) or (I′) above.

In some embodiments of the disclosure, the compound of formula (I) or (I′), or a tautomer, mesomer, racemate, enantiomer, diastereomer, or mixture thereof, or a pharmaceutically acceptable salt thereof, is a compound of formula (IV), (IV′), (IVa) or (IV′a), or a tautomer, mesomer, racemate, enantiomer, diastereomer, or mixture thereof, or a pharmaceutically acceptable salt thereof:

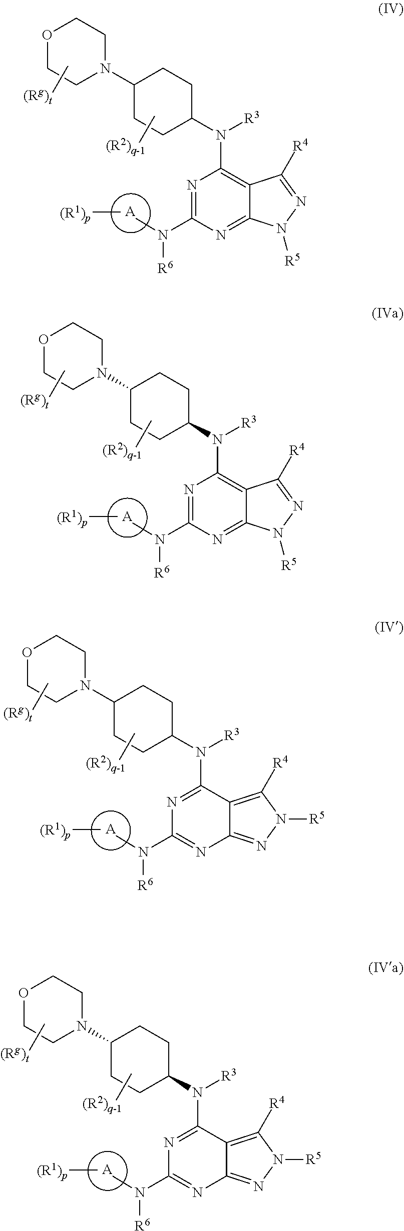

wherein:

ring A, R¹-R⁶, R^{g}, t, p and q are each as defined in formula (III) or (III′) above.

In some embodiments of the disclosure, in the compound of formula (I) or (I′), or a tautomer, mesomer, racemate, enantiomer, diastereomer, or mixture thereof, or a pharmaceutically acceptable salt thereof, ring A is pyrrolyl, thienyl, furyl, imidazolyl, thiazolyl, isothiazolyl or pyrazolyl; preferably is isothiazolyl or pyrazolyl.

In some embodiments of the disclosure, in the compound of formula (I) or (I′), or a tautomer, mesomer, racemate, enantiomer, diastereomer, or mixture thereof, or a pharmaceutically acceptable salt thereof, R¹ is identical or different and at each occurrence is independently selected from hydrogen, halogen, alkyl, alkoxy, haloalkyl, haloalkoxy, hydroxyalkyl, cyano, cycloalkyl and heterocyclyl, wherein the alkyl, cycloalkyl or heterocyclyl is optionally substituted with one or more groups independently selected from halogen, alkyl, haloalkyl, alkoxy, haloalkoxy, hydroxyalkyl, cyano, —(CH₂)ₛ—NR^{e}R^{f}; preferably R¹ is C₁₋₆ alkyl or 3-8 membered heterocyclyl; and in some embodiments more preferably R¹ is tetrahydropyranyl or methyl.

In some embodiments of the disclosure, in the compound of formula (I) or (I′), or a tautomer, mesomer, racemate, enantiomer, diastereomer, or mixture thereof, or a pharmaceutically acceptable salt thereof, R² is 3-8 membered heterocyclyl; in some embodiments preferably R² is morpholinyl.

In some embodiments of the disclosure, in the compound of formula (I) or (I′), or a tautomer, mesomer, racemate, enantiomer, diastereomer, or mixture thereof, or a pharmaceutically acceptable salt thereof, R³ is selected from hydrogen, alkyl, haloalkyl and hydroxyalkyl; in some embodiments preferably R³ is hydrogen.

In some embodiments of the disclosure, in the compound of formula (I) or (I′), or a tautomer, mesomer, racemate, enantiomer, diastereomer, or mixture thereof, or a pharmaceutically acceptable salt thereof, R⁴ is selected from hydrogen, halogen, alkyl, alkoxy, haloalkyl, haloalkoxy, hydroxyalkyl, cyano, —(CH₂)ᵣ—NR^{a}R^{b}, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyalkyl, cyano and —(CH₂)ₛ—NR^{e}R^{f};

In some embodiments, R⁴ is preferably selected from hydrogen, halogen, 6-10 membered aryl and 5-10 membered heteroaryl; more preferably R⁴ is selected from hydrogen, halogen, phenyl, pyridyl and pyrazolyl.

In some embodiments of the disclosure, in the compound of formula (I) or (I′), or a tautomer, mesomer, racemate, enantiomer, diastereomer, or mixture thereof, or a pharmaceutically acceptable salt thereof, R⁵ is selected from hydrogen, alkyl, haloalkyl, hydroxyalkyl, cycloalkyl and heterocyclyl, wherein the alkyl, cycloalkyl and heterocyclyl is optionally substituted with one or more groups independently selected from halogen, alkyl, haloalkyl, alkoxy, haloalkoxy, hydroxyalkyl, cyano and —(CH₂)ₛ—NR^{e}R^{f}; preferably R⁵ is hydrogen, C₁₋₆ alkyl or 3-8 membered heterocyclyl; further preferably R⁵ is hydrogen or 3-8 membered heterocyclyl; in some embodiments more preferably R⁵ is hydrogen or tetrahydropyranyl.

In some embodiments of the disclosure, in the compound of formula (I) or (I′), or a tautomer, mesomer, racemate, enantiomer, diastereomer, or mixture thereof, or a pharmaceutically acceptable salt thereof, R⁶ is selected from hydrogen, alkyl, haloalkyl and hydroxyalkyl; in some embodiments preferably R⁶ is hydrogen.

In some embodiments of the disclosure, in the compound of formula (I) or (I′), or a tautomer, mesomer, racemate, enantiomer, diastereomer, or mixture thereof, or a pharmaceutically acceptable salt thereof, L is a covalent bond or CH₂; in some embodiments preferably L is a covalent bond.

In some embodiments of the disclosure, in the compound of formula (I) or (I′), or a tautomer, mesomer, racemate, enantiomer, diastereomer, or mixture thereof, or a pharmaceutically acceptable salt thereof, R^{g} is identical or different and at each occurrence is independently selected from hydrogen, halogen, alkyl, haloalkyl, alkoxy, haloalkoxy, hydroxyl, hydroxyalkyl, cyano and —(CH₂)ₛ—NR^{e}R^{f}; in some embodiments preferably R^{g} is hydrogen.

In some embodiments of the disclosure, in the compound of formula (I) or (I′), or a tautomer, mesomer, racemate, enantiomer, diastereomer, or mixture thereof, or a pharmaceutically acceptable salt thereof, ring C is 3-6 membered cycloalkyl or 3-8 membered heterocyclyl; preferably is 3-8 membered heterocyclyl; more preferably morpholinyl.

In some embodiments of the disclosure, in the compound of formula (I) or (I′), or a tautomer, mesomer, racemate, enantiomer, diastereomer, or mixture thereof, or a pharmaceutically acceptable salt thereof, p is 1.

In some embodiments of the disclosure, in the compound of formula (I) or (I′), or a tautomer, mesomer, racemate, enantiomer, diastereomer, or mixture thereof, or a pharmaceutically acceptable salt thereof, q is 1.

In some embodiments of the disclosure, in the compound of formula (I) or (I′), or a tautomer, mesomer, racemate, enantiomer, diastereomer, or mixture thereof, or a pharmaceutically acceptable salt thereof, r is 0, 1 or 2, preferably is 0 or 1.

In some embodiments of the disclosure, in the compound of formula (I) or (I′), or a tautomer, mesomer, racemate, enantiomer, diastereomer, or mixture thereof, or a pharmaceutically acceptable salt thereof, s is 0, 1 or 2; preferably is 0 or 1.

In some embodiments of the disclosure, in the compound of formula (I) or (I′), or a tautomer, mesomer, racemate, enantiomer, diastereomer, or mixture thereof, or a pharmaceutically acceptable salt thereof, t is 0.

In some embodiments of the disclosure, in the compound of formula (I) or (I′), or a tautomer, mesomer, racemate, enantiomer, diastereomer, or mixture thereof, or a pharmaceutically acceptable salt thereof, R^{a}, R^{b}, R^{e} and R^{f} are identical or different and at each occurrence are independently selected from hydrogen, C₁₋₆ alkyl, C₁₋₆haloalkyl and C₁₋₆ hydroxyalkyl; or R^{a} and R^{b} together with the nitrogen to which they are attached form a 3-8 membered heterocyclyl; or R^{e} and R^{f} together with the nitrogen to which they are attached form a 3-8 membered heterocyclyl.

In some embodiments of the disclosure, in the compound of formula (I) or (I′), or a tautomer, mesomer, racemate, enantiomer, diastereomer, or mixture thereof, or a pharmaceutically acceptable salt thereof, R^{c} is C₁₋₆ alkyl.

In some embodiments of the disclosure, in the compound of formula (I) or (I′), or a tautomer, mesomer, racemate, enantiomer, diastereomer, or mixture thereof, or a pharmaceutically acceptable salt thereof, R^{d} is selected from hydrogen, C₁₋₆ alkyl, C₁₋₆ haloalkyl and C₁₋₆ hydroxyalkyl; in some embodiments preferably R^{d} is hydrogen.

In some embodiments, the disclosure provides a compound of formula (I), (I′), (III), or (III′), or a tautomer, mesomer, racemate, enantiomer, diastereomer, or mixture thereof, or a pharmaceutically acceptable salt thereof, wherein: ring A is isothiazolyl or pyrazolyl, each optionally substituted;

ring B is optionally substituted C₄-C₆ cycloalkyl;

ring C is selected from cycloalkyl, heterocyclyl, aryl, and heteroaryl, each optionally substituted;

R¹ is C₁₋₆ alkyl or 3-8 membered heterocyclyl, each optionally substituted;

R² is optionally substituted morpholinyl;

R³ is hydrogen;

R⁴ is selected from hydrogen, halogen, 6-10 membered aryl, and 5-10 membered heteroaryl, wherein the aryl and heteroaryl is optionally substituted;

R⁵ is hydrogen or optionally substituted 3-8 membered heterocyclyl; and

L is a covalent bond.

Exemplified compounds of the disclosure include, but are not limited to:

| Example | |
|---|---|
| No. | Compound structure and name |
| 1 | 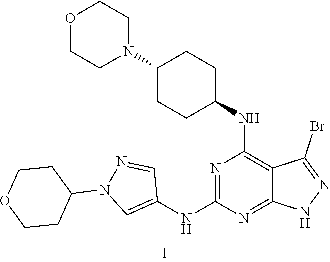 [SMILES: Brc1n[nH]c2nc(Nc3cnn(C4CCOCC4)c3)nc(N[C@H]3CC[C@H](N4CCOCC4)CC3)c12] |
| | 3-bromo-N⁴-((1r,4r)-4-morpholinocyclohexyl)-N⁶-(tetrahydro-2H- |
| | pyran-4-yl)-1H-pyrazol-4-yl)-1H-pyrazolo[3,4-d]pyrimidine-4,6- |
| | diamine 1 |
| 2 | 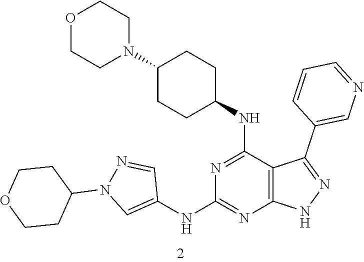 [SMILES: c1cncc(-c2n[nH]c3nc(Nc4cnn(C5CCOCC5)c4)nc(N[C@H]4CC[C@H](N5CCOCC5)CC4)c23)c1] |
| | N⁴-((1r,4r)-4-morpholinocyclohexyl)-3-(pyridin-3-yl)-N⁶-(1- |
| | (tetrahydro-2H-pyran-4-yl)-1H-pyrazol-4-yl)-1H-pyrazolo[3,4- |
| | d]pyrimidine-4,6-diamine 2 |
| 3 | 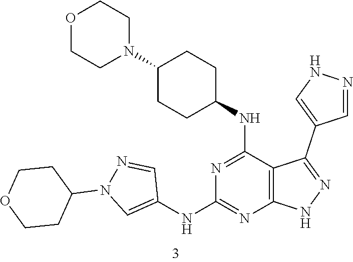 [SMILES: c1nn(C2CCOCC2)cc1Nc1nc(N[C@H]2CC[C@H](N3CCOCC3)CC2)c2c(-c3cn[nH]c3)n[nH]c2n1] |
| | N⁴-((1r,4r)-4-morpholinocyclohexyl)-3-(1H-pyrazol-4-yl)-N⁶-(1- |
| | (tetrahydro-2H-pyran-4-yl)-1H-pyrazol-4-yl)-1H-pyrazolo[3,4-d]pyrimidine-4,6- |
| | diamine 3 |
| 4 | 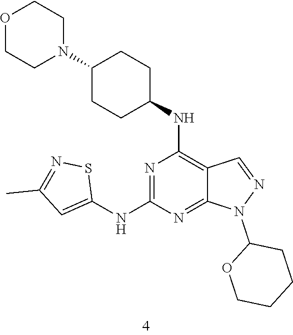 [SMILES: Cc1cc(Nc2nc(N[C@H]3CC[C@H](N4CCOCC4)CC3)c3cnn(C4CCCCO4)c3n2)sn1] |
| | N⁶-(3-methylisothiazol-5-yl)-N⁴-((1r,4r)-4-morpholinocyclohexyl)-1- |
| | (tetrahydro-2H-pyran-2-yl)-1H-pyrazolo[3,4-d]pyrimidine-4,6- |
| | diamine 4 |
| 5 | 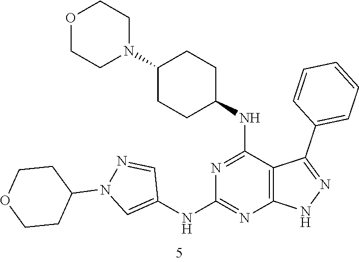 [SMILES: c1ccc(-c2n[nH]c3nc(Nc4cnn(C5CCOCC5)c4)nc(N[C@H]4CC[C@H](N5CCOCC5)CC4)c23)cc1] |
| | N⁴-((1r,4r)-4-morpholinocyclohexyl)-3-phenyl-N⁶-(1-(tetrahydro-2H- |
| | pyran-4-yl)-1H-pyrazol-4-yl)-1H-pyrazolo[3,4-d]pyrimidine-4,6- |
| | diamine 5 |
| 6 | 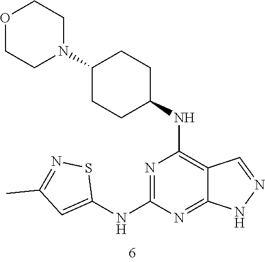 [SMILES: Cc1cc(Nc2nc(N[C@H]3CC[C@H](N4CCOCC4)CC3)c3cn[nH]c3n2)sn1] |
| | N⁶-(3-methylisothiazol-5-yl)-N⁴-((1r,4r)-4-morpholinocyclohexyl)- |
| | 1H-pyrazolo[3,4-d]pyrimidine-4,6-diamine 6 |
| 7d | 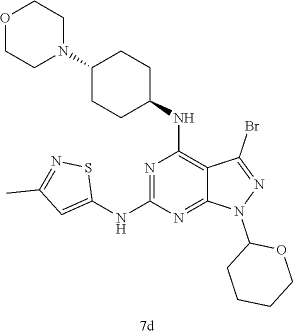 [SMILES: Cc1cc(Nc2nc(N[C@H]3CC[C@H](N4CCOCC4)CC3)c3c(Br)nn(C4CCCCO4)c3n2)sn1] |
| | 3-bromo-N⁶-(3-methylisothiazol-5-yl)-N⁴-((1r,4r)-4- |
| | morpholinocyclohexyl)-1-(tetrahydro-2H-pyran-2-yl)-1H- |
| | pyrazolo[3,4-d]pyrimidine-4,6-diamine 7d |
| 7 | 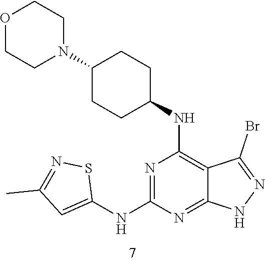 [SMILES: Cc1cc(Nc2nc(N[C@H]3CC[C@H](N4CCOCC4)CC3)c3c(Br)n[nH]c3n2)sn1] |
| | 3-bromo-N⁶-(3-methylisothiazol-5-yl)-N⁴-((1r,4r)-4- |
| | morpholinocyclohexyl)-1H-pyrazolo[3,4-d]pyrimidine-4,6-diamine 7 |

or a tautomer, mesomer, racemate, enantiomer, diastereomer, or mixture thereof, or a pharmaceutically acceptable salt thereof.

In one aspect, the present disclosure provides a compound of formula (IA1) or (I′A1), or a tautomer, mesomer, racemate, enantiomer, diastereomer, or mixture thereof, or pharmaceutically acceptable salt thereof:

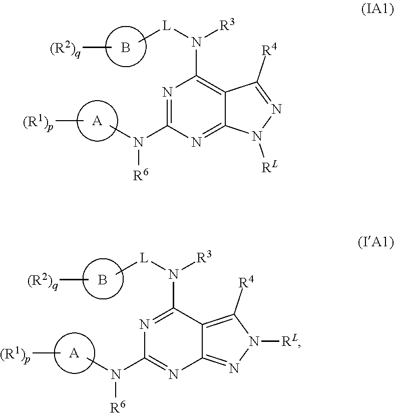

wherein R^{L} is a protecting group, preferably amino protecting group, more preferably tetrahydropyranyl, and further preferably tetrahydropyran-2-yl; and

ring A, ring B, L, R¹ to R⁴, R⁵, p and q are each as defined in formula (I) or (I′).

In one aspect, the present disclosure provides a compound of formula (IA2) or (I′A2), or a tautomer, mesomer, racemate, enantiomer, diastereomer, or mixture thereof, or pharmaceutically acceptable salt thereof:

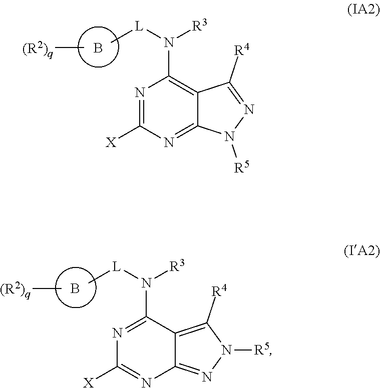

wherein X is halogen; and

ring B, L, R² to R⁵ and q are each as defined in formula (I) or (I′).

In one aspect, the present disclosure provides a compound of formula (IA3) or (I′A3), or a tautomer, mesomer, racemate, enantiomer, diastereomer, or mixture thereof, or pharmaceutically acceptable salt thereof:

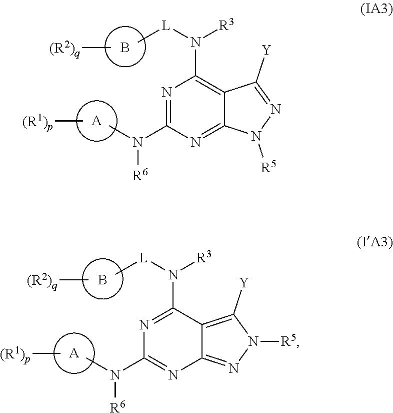

wherein Y is halogen or OTf; and

ring A, ring B, L, R¹ to R³, R⁵, R⁶, p and q are each as defined in formula (I) or (I′).

In one aspect, the present disclosure provides a compound of formula (IIA1) or (II′A1), or a tautomer, mesomer, racemate, enantiomer, diastereomer, or mixture thereof, or pharmaceutically acceptable salt thereof:

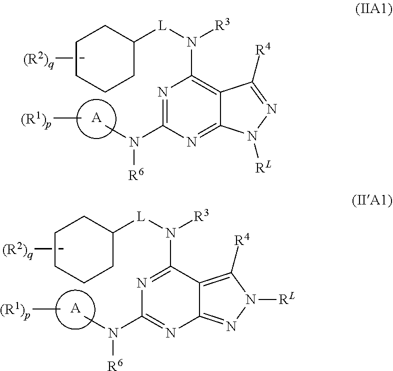

wherein R^{L} is a protecting group; preferably is amino protecting group; more preferably is tetrahydropyranyl; further preferably is tetrahydropyran-2-yl; and

ring A, L, R¹ to R⁴, R⁶, p and q are each as defined in formula (II) or (II′).

In one aspect, the present disclosure provides a compound of formula (IIA2) or (II′A2), or a tautomer, mesomer, racemate, enantiomer, diastereomer, or mixture thereof, or pharmaceutically acceptable salt thereof:

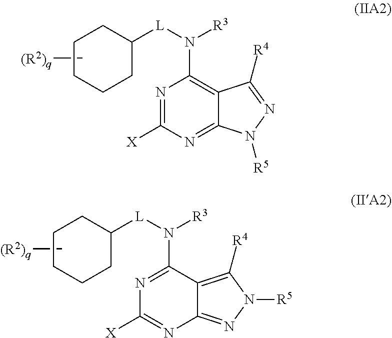

wherein X is halogen;

L, R² to R⁵ and q are each as defined in formula (II) or (II′); and

provided that at least one R² is selected from heterocyclyl, aryl, heteroaryl and cycloalkyl.

In one aspect, the present disclosure provides a compound of formula (IIA3) or (II′A3), or a tautomer, mesomer, racemate, enantiomer, diastereomer, or mixture thereof, or pharmaceutically acceptable salt thereof:

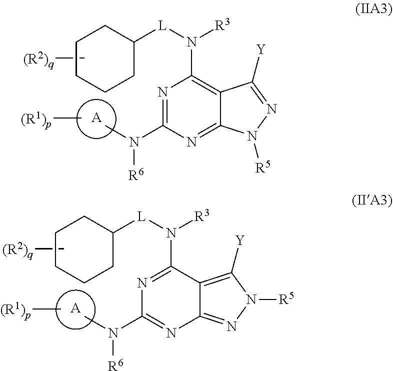

wherein Y is halogen or OTf; and

ring A, L, R¹ to R³, R⁵, R⁶, p and q are each as defined in formula (II) or (II′).

In one aspect, the present disclosure provides a compound of formula (IIIA1) or (III′A1), or a tautomer, mesomer, racemate, enantiomer, diastereomer, or mixture thereof, or pharmaceutically acceptable salt thereof:

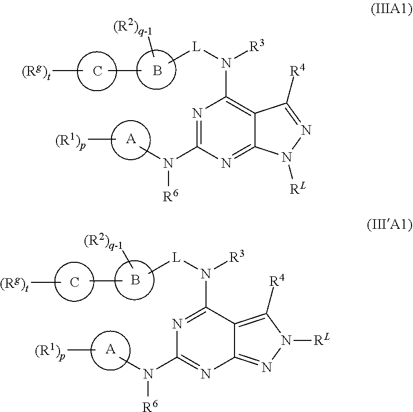

wherein R^{L} is a protecting group, preferably an amino protecting group, more preferably tetrahydropyranyl, and further preferably tetrahydropyran-2-yl; and

ring A, ring B, ring C, L, R¹ to R⁴, R⁶, R^{g}, t, p and q are each as defined in formula (III) or (III′).

In one aspect, the present disclosure provides a compound of formula (IIIA2) or (III′A2), or a tautomer, mesomer, racemate, enantiomer, diastereomer, or mixture thereof, or pharmaceutically acceptable salt thereof:

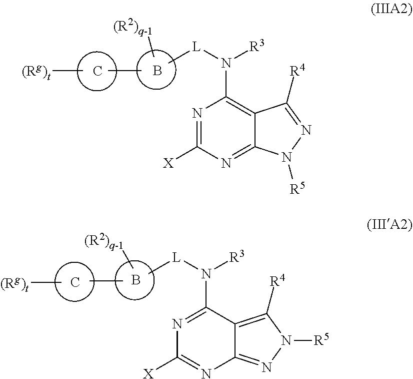

wherein:

X is halogen;

ring B is a heterocyclyl or C₄₋₆ cycloalkyl;

ring C is selected from heterocyclyl, aryl, heteroaryl and C₄₋₆ cycloalkyl;

R^{g} at each occurrence is identical or different and is independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl; and L, R²-R⁵, R^{e}, R^{f}, s, t and q are each as defined in formula (III) or (III′).

In one aspect, the present disclosure provides a compound of formula (IIIA3) or (II′A3), or a tautomer, mesomer, racemate, enantiomer, diastereomer, or mixture thereof, or pharmaceutically acceptable salt thereof:

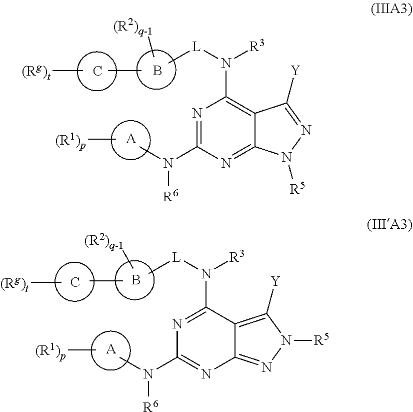

wherein Y is halogen or OTf; and

ring A, ring B, ring C, L, R¹ to R³, R⁵, R⁶, R^{g}, t, p and q are each as defined in formula (III) or (III′).

In one aspect, the present disclosure provides a compound of formula (IVA1), (IV′A1), (IVaA1) or formula (IV′aA1), or a tautomer, mesomer, racemate, enantiomer, diastereomer, or mixture thereof, or pharmaceutically acceptable salt thereof:

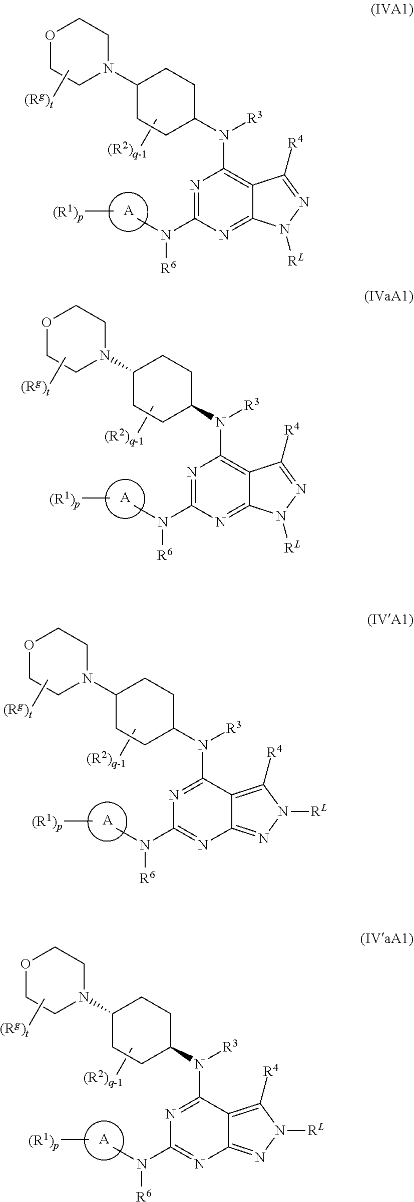

wherein R^{L} is a protecting group, preferably an amino protecting group, more preferably tetrahydropyranyl, and further preferably tetrahydropyran-2-yl; and

ring A, R¹ to R⁴, R⁶, R^{g}, t, p and q are each as defined in formula (IV) or (IV′).

In one aspect, the present disclosure provides a compound of formula (IVA2), (IV′A2), (IVaA2) or formula (IV′aA2), or a tautomer, mesomer, racemate, enantiomer, diastereomer, or mixture thereof, or pharmaceutically acceptable salt thereof:

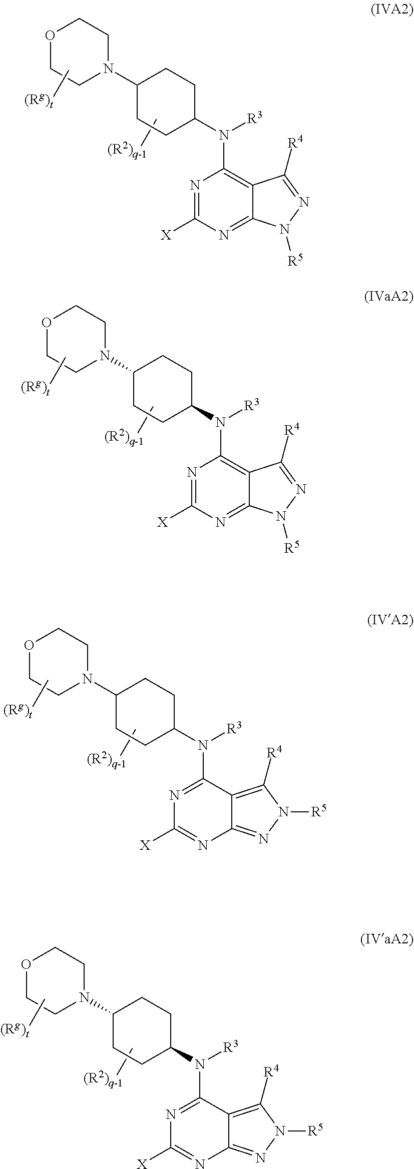

wherein X is halogen; and

R² to R⁵, R^{g}, t and q are each as defined in formula (IV) or (IV′).

In one aspect, the present disclosure provides a compound of formula (IVA3), (IV′A3), (IVaA3) or formula (IV′aA3), or a tautomer, mesomer, racemate, enantiomer, diastereomer, or mixture thereof, or pharmaceutically acceptable salt thereof:

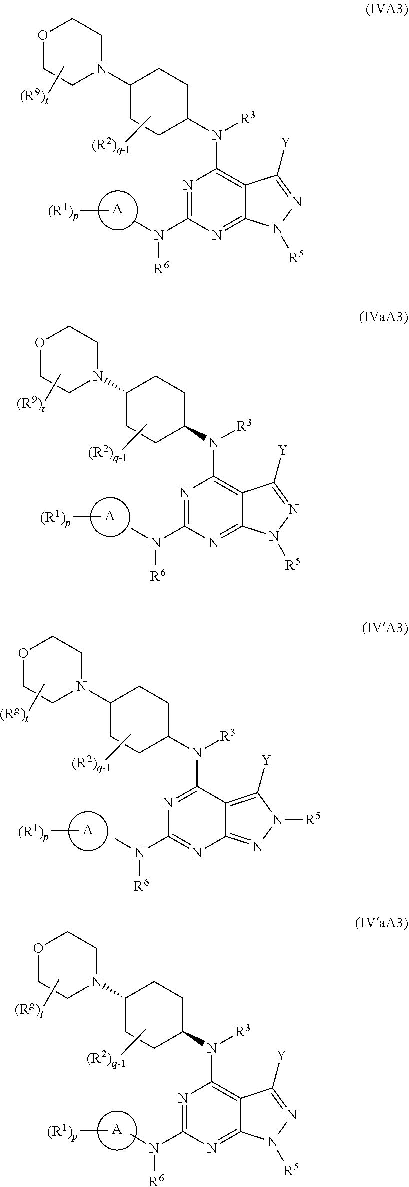

wherein Y is halogen or OTf, and

ring A, R¹ to R³, R⁵, R⁶, R^{g}, t, p and q are each as defined in formula (IV) or (IV′).

Exemplified compounds of the disclosure include, but are not limited to:

| Example | |
|---|---|
| No. | Compound structure and name |
| 1c | 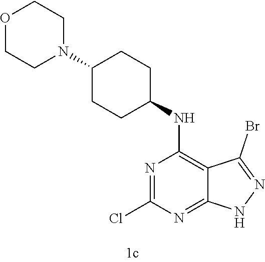 [SMILES: Clc1nc(N[C@H]2CC[C@H](N3CCOCC3)CC2)c2c(Br)n[nH]c2n1] |
| | 3-bromo-6-chloro-N-((1r,4r)-4-morpholinocyclohexyl)-1H- |
| | pyrazolo[3,4-d]pyrimidin-4-amine 1c |
| 4c | 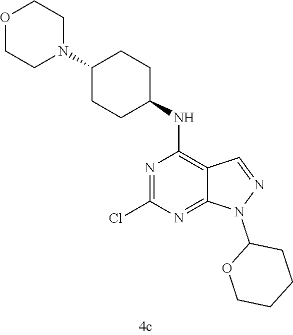 [SMILES: Clc1nc(N[C@H]2CC[C@H](N3CCOCC3)CC2)c2cnn(C3CCCCO3)c2n1] |
| | 6-chloro-N-((1r,4r)-4-morpholinocyclohexyl)-1-(tetrahydro-2- |
| | pyran-2-yl)-1H-pyrazolo[3,4-d]pyrimidin-4-amine 4c |

or a tautomer, mesomer, racemate, enantiomer, diastereomer, or mixture thereof, or a pharmaceutically acceptable salt thereof.

As a person of ordinary skill in the art would understand, any and all reasonable combinations of the embodiments disclosed herein, especially with regard to the definitions of any substituents, e.g., ring A, ring B, ring C, L, R¹-R⁶, R^{f}, R^{e}, R^{g}, s, t, p and q, or the like, in the compounds of formulae (I)-(IV), (I′)-(IV′), and intermediate or precursor compounds thereof, or the like, are all encompassed by the present invention.

In another aspect, this disclosure provides a preparation process of a compound of formula (I) or (I′), or a tautomer, mesomer, racemate, enantiomer, diastereomer, or mixture thereof, or a pharmaceutically acceptable salt thereof, the preparation process comprising the steps of:

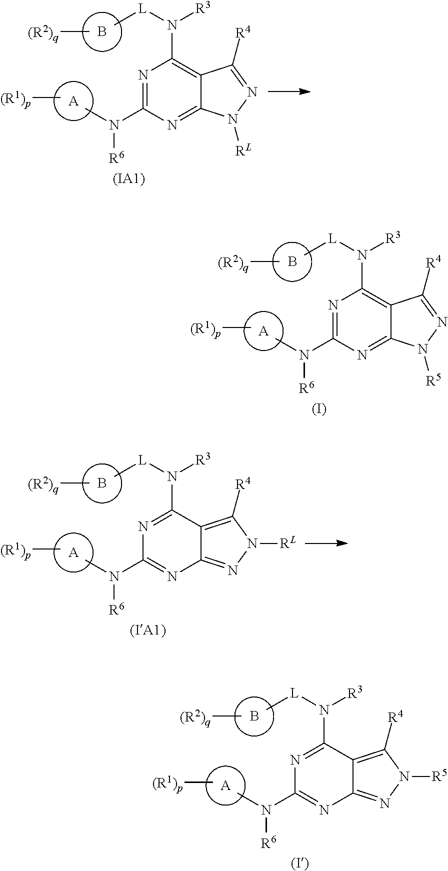

the compound of formula (IA1) or (I′A1) remove the group R^{L} to obtain the compound of formula (I) or (I′), wherein:

R^{L} is a protecting group, preferably tetrahydropyranyl;

R⁵ is H; and

ring A, ring B, L, R¹ to R⁴, R⁶, p and q are each as defined in formula (I) or (I′).

In another aspect, this disclosure provides a preparation process of a compound of formula (I) or (I′), or a tautomer, mesomer, racemate, enantiomer, diastereomer, or mixture thereof, or a pharmaceutically acceptable salt thereof, the preparation process comprising the steps of:

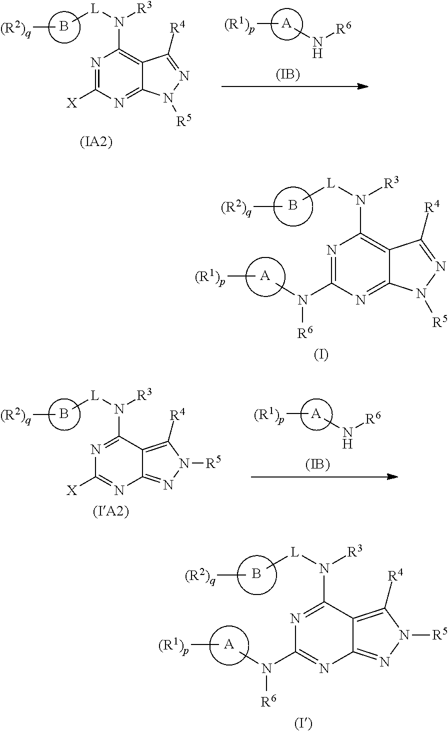

reacting a compound of formula (IA2) or (I′A2) with a compound of formula (IB) or a salt thereof to obtain the compound of formula (I) or (I′), wherein:

X is halogen;

the salt of the compound (IB) preferably is hydrochloride; and

ring A, ring B, L, R¹ to R⁶, p and q are each as defined in formula (I) or (I′).

In another aspect, this disclosure provides a preparation process of a compound of formula (I) or (I′), or a tautomer, mesomer, racemate, enantiomer, diastereomer, or mixture thereof, or a pharmaceutically acceptable salt thereof, the preparation process comprising the steps of:

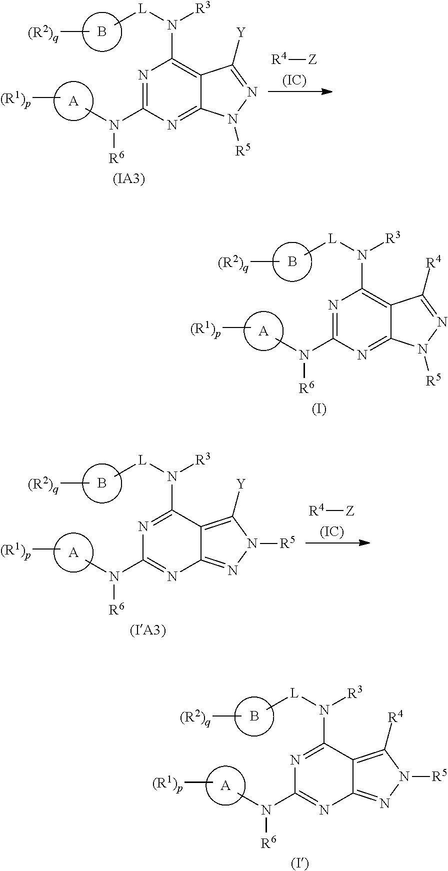

reacting a compound of formula (IA3) or (I′A3) with a compound of formula (IC) to obtain the compound of formula (I) or (I′), wherein:

Y is halogen or OTf;

Z is selected from

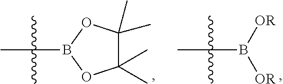

—MgX′, —ZnY′, BF₃—, —Sn(R)₃, —Si(R)₃, and hydrogen;

R is hydrogen or alkyl;

X′ and Y′ are halogen;

provided that only when R⁴ is —NR^{a}R^{b}, alkoxy or haloalkoxy, Z is hydrogen; and

ring A, ring B, L, R¹ to R⁶, R^{a}, R^{b}, p and q are each as defined in formula (I) or (I′).

In another aspect, this disclosure provides a preparation process of a compound of formula (II) or (II′), or a tautomer, mesomer, racemate, enantiomer, diastereomer, or mixture thereof, or a pharmaceutically acceptable salt thereof, the preparation process comprising the steps of:

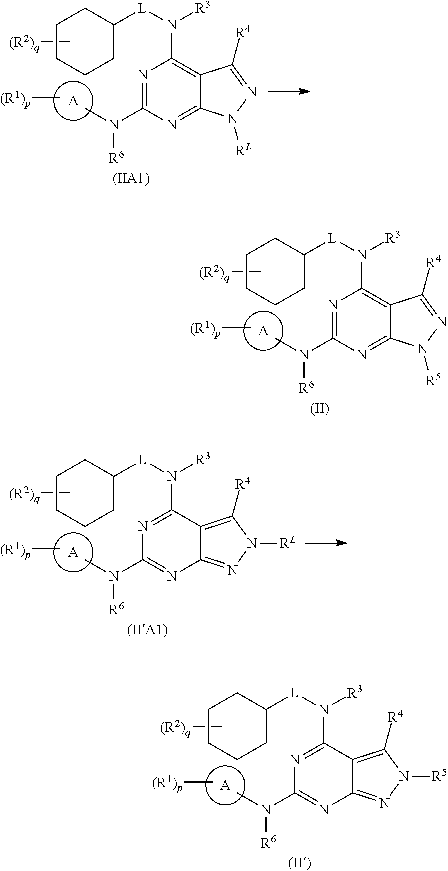

removing the group R^{L} from the compound of formula (IIA1) or (II′A1) to obtain the compound of formula (II) or (II′), wherein:

R^{L} is a protecting group, preferably tetrahydropyranyl;

R⁵ is H; and

ring A, L, R¹ to R⁴, R⁶, p and q are each as defined in formula (II) or (II′).

In another aspect, this disclosure provides a preparation process of a compound of formula (II) or (II′), or a tautomer, mesomer, racemate, enantiomer, diastereomer, or mixture thereof, or a pharmaceutically acceptable salt thereof, the preparation process comprising the steps of:

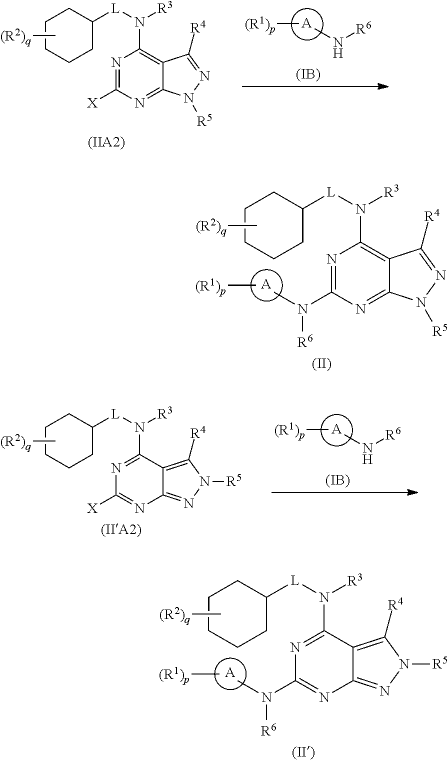

reacting a compound of formula (IIA2) or (II′A2) with a compound of formula (IB) or a salt thereof to obtain the compound of formula (II) or (II′), wherein:

X is halogen;

the salt of the compound (IB) preferably is hydrochloride; and

ring A, L, R¹ to R⁶, p and q are each as defined in formula (II) or (II′).

In another aspect, this disclosure provides a preparation process of a compound of formula (II) or (II′), or a tautomer, mesomer, racemate, enantiomer, diastereomer, or mixture thereof, or a pharmaceutically acceptable salt thereof, the preparation process comprising the steps of:

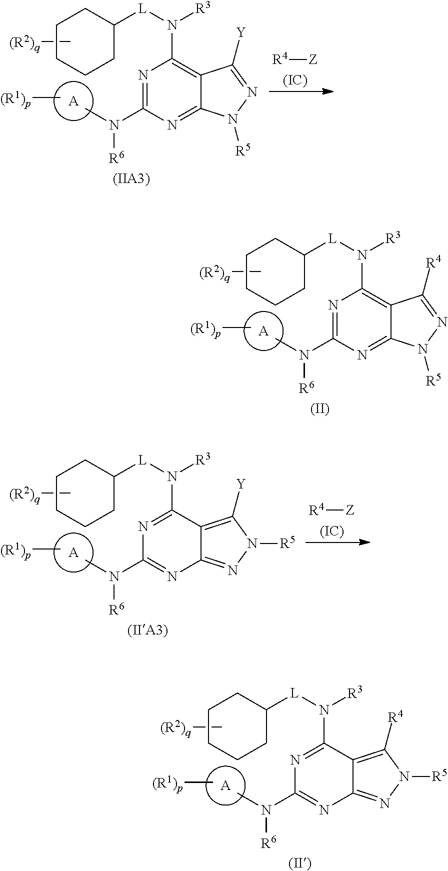

reacting a compound of formula (IIA3) or (I′A3) with a compound of formula (IC) to obtain the compound of formula (II) or (II′), wherein:

Y is halogen or OTf

Z is selected from

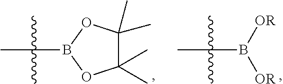

—MgX′, —ZnY′, BF₃—, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen; and

ring A, L, R¹ to R⁶, R^{a}, R^{b}, p and q are each as defined in formula (II) or (II′).

In another aspect, this disclosure provides a preparation process of a compound of formula (III) or (III′), or a tautomer, mesomer, racemate, enantiomer, diastereomer, or mixture thereof, or a pharmaceutically acceptable salt thereof, the preparation process comprising the steps of:

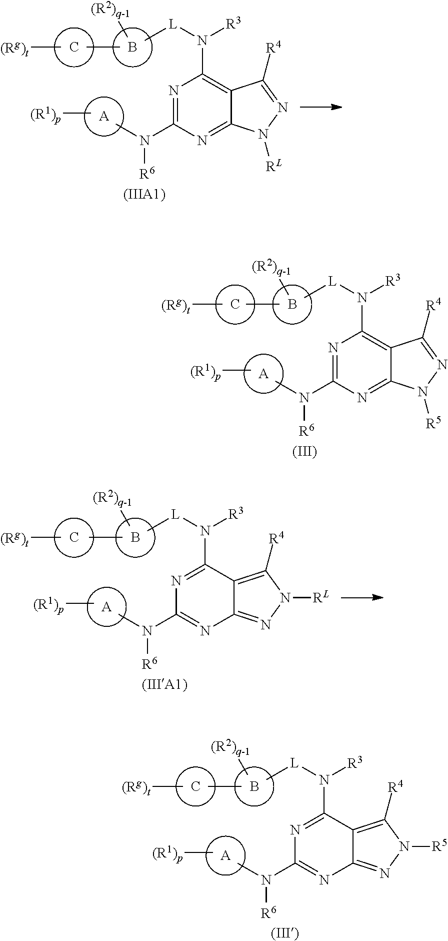

removing the group R^{L} from the compound of formula (IIIA1) or (III′A1) to obtain the compound of formula (III) or (III′), wherein:

R^{L} is a protecting group, preferably tetrahydropyranyl;

R⁵ is H; and

ring A, ring B, ring C, L, R¹ to R⁴, R⁶, R^{g}, t, p and q are each as defined in formula (III) or (III′).

In another aspect, this disclosure provides a preparation process of a compound of formula (III) or (III′), or a tautomer, mesomer, racemate, enantiomer, diastereomer, or mixture thereof, or a pharmaceutically acceptable salt thereof, the preparation process comprising the steps of:

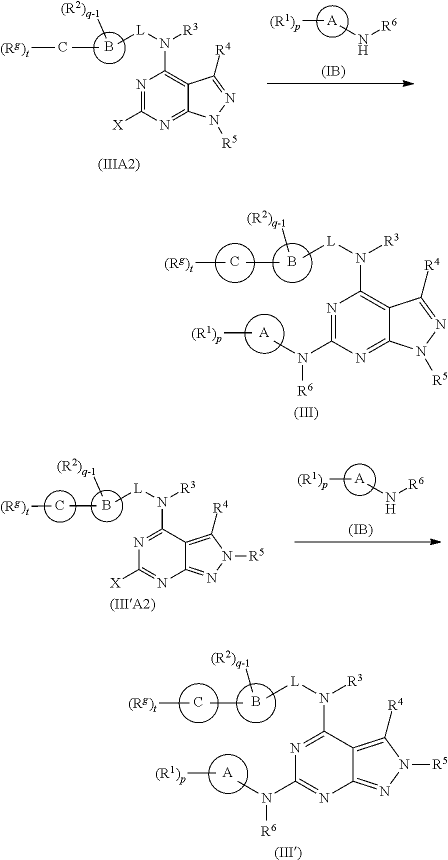

reacting a compound of formula (IIIA2) or (III′A2) with a compound of formula (IB) or a salt thereof to obtain the compound of formula (III) or (III′), wherein:

X is halogen;

the salt of the compound (IB) preferably is hydrochloride; and

ring A, ring B, ring C, L, R¹ to R⁶, R^{g}, t, p and q are each as defined in formula (III) or (III′).

In another aspect, this disclosure provides a preparation process of a compound of formula (III) or (III′), or a tautomer, mesomer, racemate, enantiomer, diastereomer, or mixture thereof, or a pharmaceutically acceptable salt thereof, the preparation process comprising the steps of:

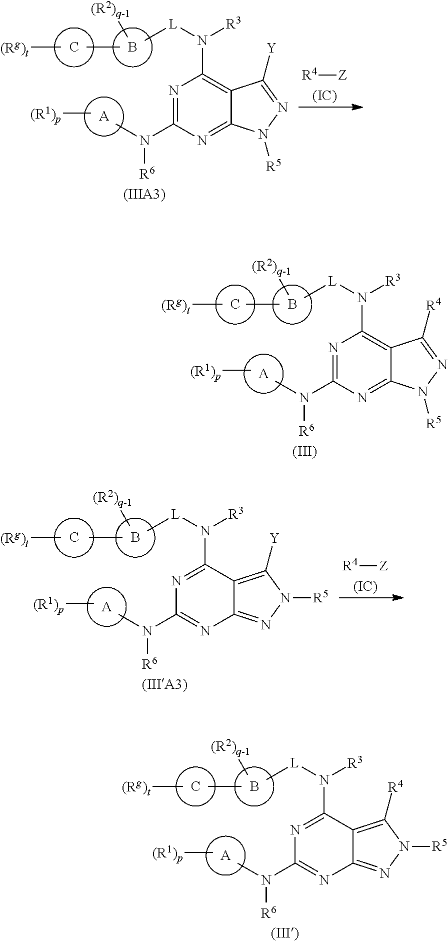

reacting a compound of formula (IIIA3) or (III′A3) with a compound of formula (IC) to obtain the compound of formula (III) or (III′), wherein:

Y is halogen or OTf;

Z is selected from

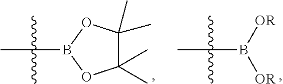

—MgX′, —ZnY′, BF₃, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen; and

ring A, ring B, ring C, L, R¹ to R⁶, R^{a}, R^{b}, R^{g}, t, p and q are each as defined in formula (III) or ((I′).

In another aspect, this disclosure provides a preparation process of a compound of formula (IV), (IV′), (IVa) or (IV′a), or a tautomer, mesomer, racemate, enantiomer, diastereomer, or mixture thereof, or a pharmaceutically acceptable salt thereof, the preparation process comprising the steps of:

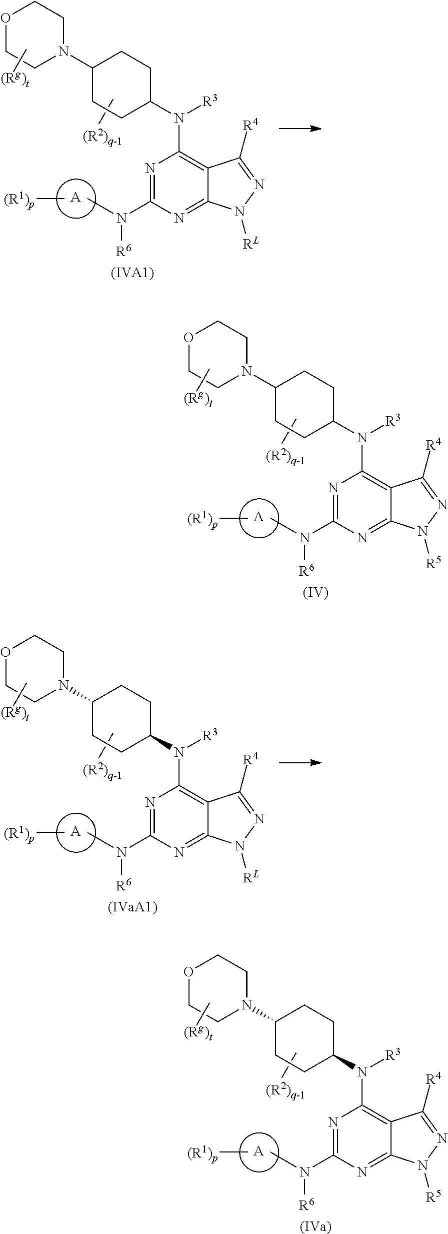

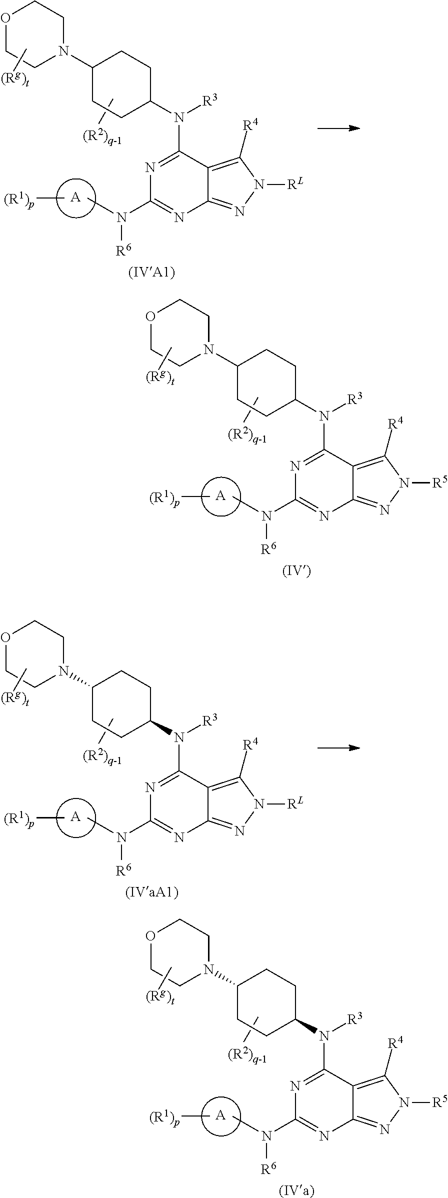

removing the group R^{L} from the compound of formula (IVA1), (IV′A1), (IVaA1) or (IV′aA1) to obtain the compound of formula (IV), (IV′), (IVa) or (IV′a), wherein:

R^{L} is a protecting group, preferably tetrahydropyranyl;

R⁵ is H and

ring A, R¹ to R⁴, R⁶, R^{g}, t, p and q are each as defined in formula (IV) or (IV′).

In another aspect, this disclosure provides a preparation process of a compound of formula (IV), (IV′), (IVa) or (IV′a), or a tautomer, mesomer, racemate, enantiomer, diastereomer, or mixture thereof, or a pharmaceutically acceptable salt thereof, the preparation process comprising the steps of:

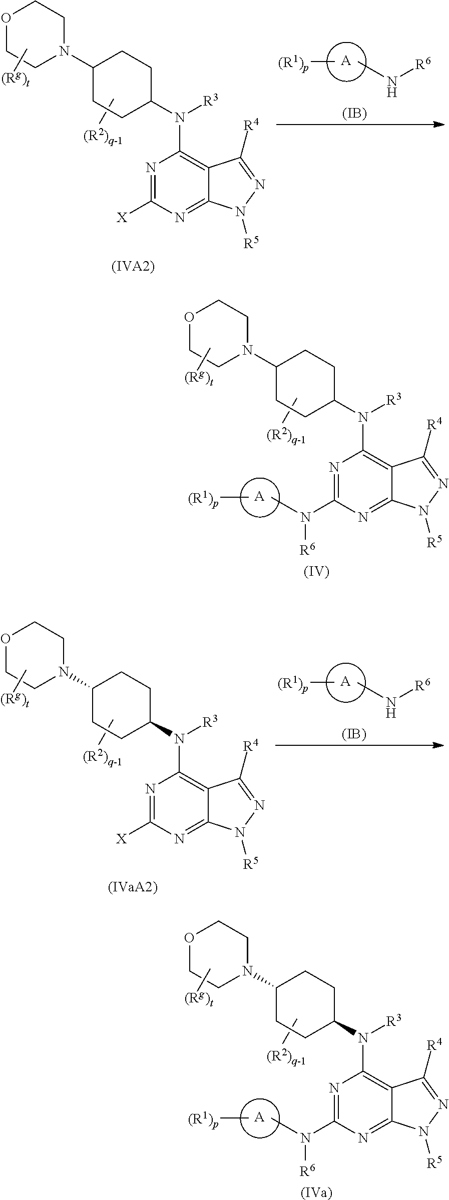

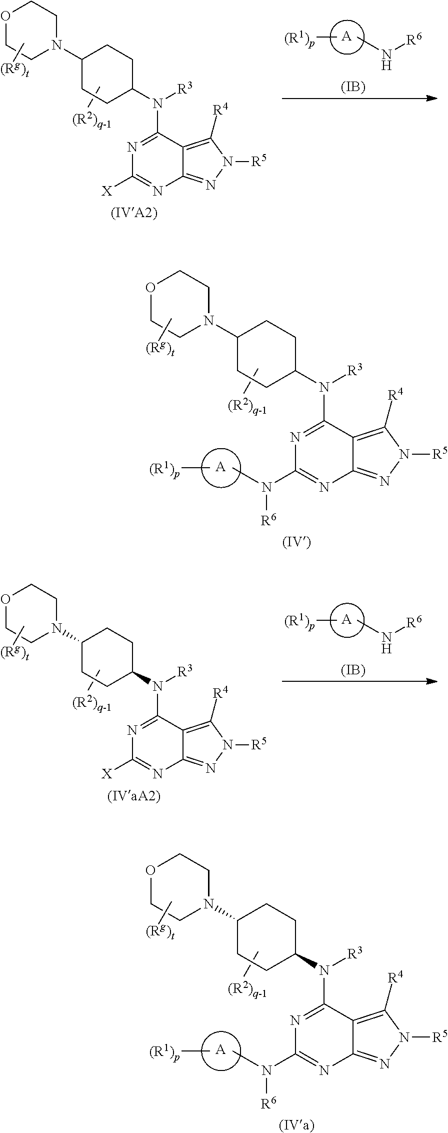

reacting a compound of formula (IVA2), (IV′A2), (IVaA2) or (IV′aA2) with a compound of formula (IB) or a salt thereof to obtain the compound of formula (IV), (IV′), (IVa) or (IV′a), wherein:

X is halogen;

the salt of the compound (IB) preferably is hydrochloride; and

ring A, R¹-R⁶, R^{g}, t, p and q are each as defined in formula (IV) or (IV′).

In another aspect, this disclosure provides a preparation process of a compound of formula (IV), (IV′), (IVa) or (IV′a), or a tautomer, mesomer, racemate, enantiomer, diastereomer, or mixture thereof, or a pharmaceutically acceptable salt thereof, the preparation process comprising the steps of:

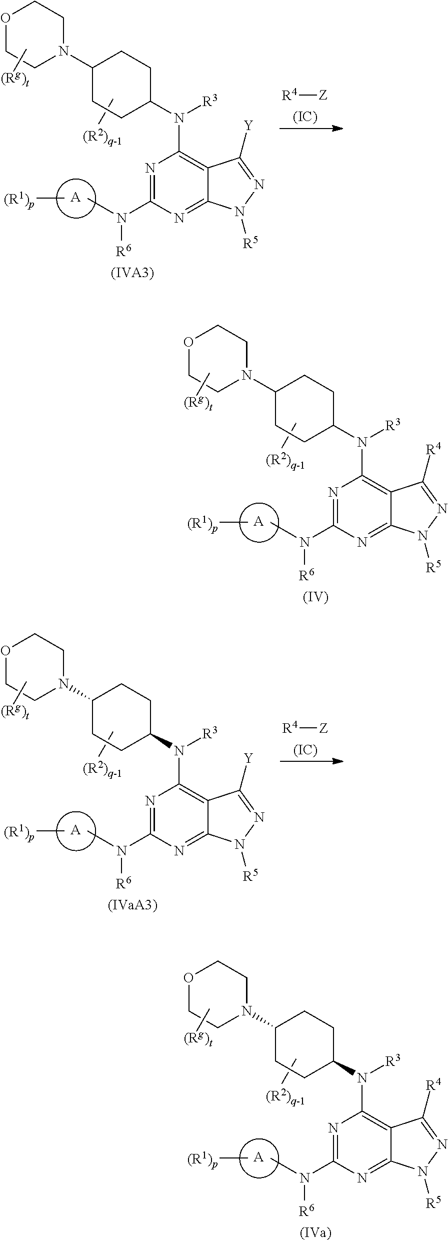

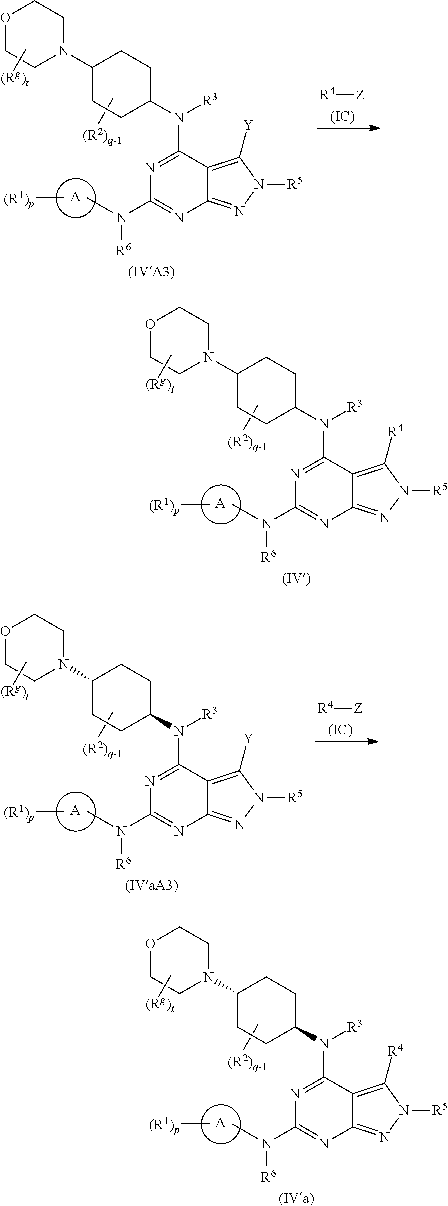

reacting a compound of formula (IVA3), (IV′A3), (IVaA3) or (IV′aA3) with a compound of formula (IC) to obtain the compound of formula (IV), (IV′), (IVa) or (IV′a), wherein:

Y is halogen or OTf;

Z is selected from

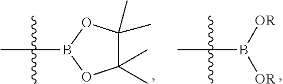

—MgX′, —ZnY′, BF₃, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen; and

ring A, R¹-R⁶, R^{a}, R^{b}, R^{g}, t, p and q are each as defined in formula (IV) or (IV′).

The present disclosure also provides a pharmaceutical composition, comprising a compound of formula (I) or (I′), or a tautomer, mesomer, racemate, enantiomer, diastereomer, or mixture thereof, or a pharmaceutically acceptable salt thereof, and a pharmaceutically acceptable carrier.

The present disclosure also provides a pharmaceutical composition, comprising a therapeutically effective amount of a compound of formula (I) or (I′), or a tautomer, mesomer, racemate, enantiomer, diastereomer, or mixture thereof, or a pharmaceutically acceptable salt thereof, and one or more pharmaceutically acceptable carriers, diluents or other excipients.

The present disclosure also provides a method of inhibiting IRAK protein kinase, comprising administering to a subject in need thereof a therapeutically effective amount of the compound of formula (I) or (I′), or a tautomer, mesomer, racemate, enantiomer, diastereomer, or mixture thereof, or a pharmaceutically acceptable salt thereof, or the pharmaceutical composition containing the compound; preferably the IRAK protein kinase is an IRAK-4 protein kinase.

The present disclosure also provides a method of preventing and/or treating IRAK-mediated disorder, disease, or condition, comprising a step of administering to a subject in need thereof a therapeutically effective amount of the compound of formula (I) or (I′), or a tautomer, mesomer, racemate, enantiomer, diastereomer, or mixture thereof, or a pharmaceutically acceptable salt thereof, or pharmaceutical composition containing the compound.

The present disclosure also provides a method of preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder, the method comprising a step of administering to a subject in need thereof a therapeutically effective amount of the compound of formula (I) or (I′), or a tautomer, mesomer, racemate, enantiomer, diastereomer, or mixture thereof, or a pharmaceutically acceptable salt thereof, or pharmaceutical composition containing the compound.

In other words, the present disclosure also provides use of a compound of formula (I) or (I′), or a tautomer, mesomer, racemate, enantiomer, diastereomer, or mixture thereof, or a pharmaceutically acceptable salt thereof, in the preparation of a medicament for the inhibition of IRAK protein kinase; preferably the IRAK protein kinase is an IRAK-4 protein kinase.

In other words, the present disclosure also relates to use of a compound of formula (I) or (I′) or a tautomer, mesomer, racemate, enantiomer, diastereomer, or mixture thereof, or a pharmaceutically acceptable salt thereof, in the preparation of a medicament for preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The present disclosure also relates to the combination of the compound of formula (I) or (I′), or a tautomer, mesomer, racemate, enantiomer, diastereomer, or mixture thereof, or a pharmaceutically acceptable salt thereof, with a second therapeutic agent for use in preventing and/or treating IRAK-mediated disorder, disease, or condition.

The present disclosure also relates to the combination of the compound of formula (I) or (I′), or a tautomer, mesomer, racemate, enantiomer, diastereomer, or mixture thereof, or a pharmaceutically acceptable salt thereof, with a second therapeutic agent for use in preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The “cancer” or “proliferative disorder” mentioned includes, but is not limited to, a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABCDLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulmemia (WM), splenic marginal zone lymphoma, multiple myeloma, plasmacytoma, and intravascular large B-cell lymphoma).

The “MyD88 driven disorder” mentioned includes, but is not limited to, ABC DLBCL, Waldenstrom's macroglobulmemia, Hodgkin's lymphoma, primary cutaneous T-cell lymphoma and chronic lymphocytic leukemia; and/or the IL-1 driven disorder is Smoldering of indolent multiple myeloma.

The “IL-1” driven disorder mentioned is Smoldering of indolent multiple myeloma.

The “neurodegenerative disease” mentioned includes, but is not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

The “inflammatory disorder” refers to conditions of the eye such as ocular allergy, conjunctivitis, keratoconjunctivitis sicca, and vernal conjunctivitis; diseases affecting the nose including allergic rhinitis; and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospirosis renal disease, glaucoma, retinal disease, ageing, headache, pain, complex regional pain syndrome, cardiac hypertrophy, musclewasting, catabolic disorders, obesity, fetal growth retardation, hypercholesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, vulvitis, alopecia areata, erythema multiforma, dermatitis herpetiformis, scleroderma, vitiligo, hypersensitivity angiitis, urticaria, bullous pemphigoid, Pemphigus vulgaris, Pemphigus foliaceus, paraneoplastic pemphigus, epidermolysis bullosa acquisita, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration. The compounds of this disclosure can also be formulated as sustained release dosage forms.

Suitable dosage forms include, but are not limited to, a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water-soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS.™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound of formula (I) or (I′) or the type of pharmaceutically acceptable salt thereof can be verified by traditional therapeutic regimens.

Unless otherwise stated, the terms used in the specification and claims have the meanings described below.

“Alkyl” refers to a saturated aliphatic hydrocarbon group including C₁-C₂₀ straight chain and branched chain groups. Preferably an alkyl group is an alkyl having 1 to 12 (for example 1, 2, 3, 4, 5, 6, 7, 8, 9, 10, 11 or 12) carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 (for example 1, 2, 3, 4, 5 or 6) carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more, preferably one to five, and sometimes more preferably one to three, groups independently selected from alkyl, halogen, alkoxy, alkenyl, alkynyl, alkylthio, alkylamino, thiol, hydroxyl, nitro, cyano, amino, cycloalkyl, heterocyclyl, aryl, heteroaryl, cycloalkoxy, cycloalkylthio, heterocyclylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, preferably one to five, and sometimes more preferably one to three, group(s) independently selected from alkyl, halogen, alkoxy, alkenyl, alkynyl, alkylthio, alkylamino, thiol, hydroxyl, nitro, cyano, amino, cycloalkyl, heterocyclyl, aryl, heteroaryl, cycloalkoxy, cycloalkylthio, heterocyclylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and most preferably C₂₋₆ alkynyl. The alkynyl group can be substituted or unsubstituted. When substituted, the substituent group(s) is preferably one or more, preferably one to five, and sometimes more preferably one to three, group(s) independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 (for example 1, 2, 3, 4, 5, 6, 7, 8, 9, 10, 11 or 12) carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, preferably one to five, and sometimes more preferably one to three, group(s) independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂—etc. The alkenylene group can be substituted or unsubstituted. When substituted, the substituent group(s) is preferably one or more, preferably one to five, and sometimes more preferably one to three, group(s) independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 (for example 3, 4, 5, 6, 7 or 8) carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more, preferably one to three, double bonds. Preferably a spiro cycloalkyl is 6 to 14 membered, and more preferably 7 to 10 (for example 7, 8, 9 or 10) membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following groups:

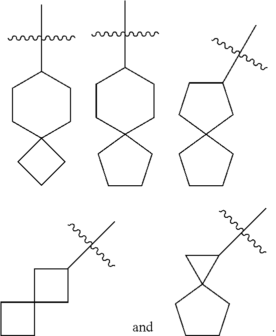

“Fused Cycloalkyl” refers to a 5 to 20 membered polycyclic hydrocarbon group, wherein each ring in the system shares an adjacent pair of carbon atoms with another ring, wherein one or more rings can contain one or more, preferably one to three, double bonds. Preferably, a fused cycloalkyl group is 6 to 14 membered, more preferably 7 to 10 (for example 7, 8, 9 or 10) membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused cycloalkyl. Representative examples of fused cycloalkyls include, but are not limited to, the following groups:

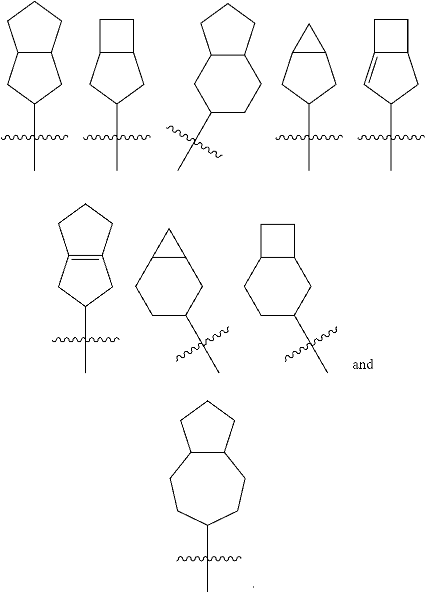

“Bridged Cycloalkyl” refers to a 5 to 20 membered polycyclic hydrocarbon group, wherein every two rings in the system share two disconnected carbon atoms. The rings can have one or more, preferably one to three, double bonds, but have no completely conjugated pi-electron system. Preferably, a bridged cycloalkyl is 6 to 14 membered, and more preferably 7 to 10 (for example 7, 8, 9 or 10) membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

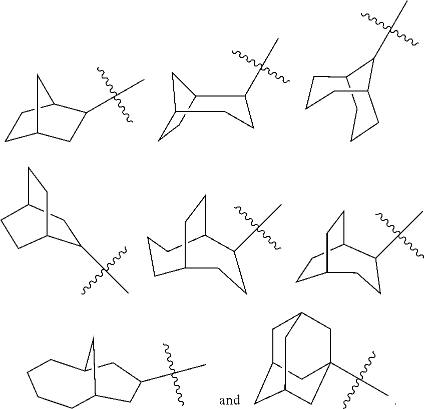

The cycloalkyl include the cycloalkyl said above which fused to the ring of an aryl, heteroaryl or heterocyclyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, preferably one to five, and sometimes more preferably one to three, groups independently selected from alkyl, halogen, alkoxy, alkenyl, alkynyl, alkylthio, alkylamino, thiol, hydroxyl, nitro, cyano, amino, cycloalkyl, heterocyclyl, aryl, heteroaryl, cycloalkoxy, cycloalkylthio, heterocyclylthio and oxo group.

“Heterocyclyl” refers to a 3 to 20 membered saturated and/or partially unsaturated monocyclic or polycyclic hydrocarbon group having one or more, preferably one to five, and sometimes more preferably one to three, heteroatoms selected from N, O, S, S(O) and S(O)₂ as ring atoms, but excluding —O—O—, —O—S— or —S—S— in the ring, the remaining ring atoms being C. Preferably, heterocyclyl is a 3 to 12 (for example 3, 4, 5, 6, 7, 8, 9, 10, 11 or 12) membered having 1 to 4 (for example 1, 2, 3 or 4) heteroatoms; more preferably a 3 to 8 (for example 3, 4, 5, 6, 7 or 8) membered having 1 to 3 (for example 1, 2 or 3) heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, preferably one to five, and sometimes more preferably one to three, heteroatoms selected from N, O, S, S(O) and S(O)₂ as ring atoms, the remaining ring atoms being C, wherein one or more, preferably one to three, rings can contain one or more double bonds. Preferably a spiro heterocyclyl is 6 to 14 membered, and more preferably 7 to 10 (for example 7, 8, 9 or 10) membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following groups:

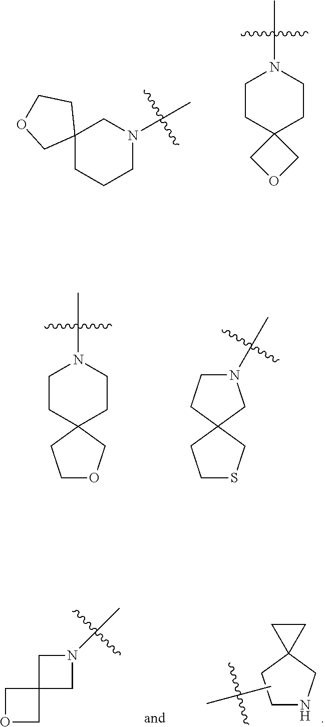

“Fused Heterocyclyl” refers to a 5 to 20 membered polycyclic heterocyclyl group, wherein each ring in the system shares an adjacent pair of carbon atoms with the other ring, wherein one or more rings can contain one or more, preferably one to three, double bonds, and wherein said rings have one or more heteroatoms selected from N, O, S, S(O) and S(O)₂ as ring atoms, the remaining ring atoms being C. Preferably a fused heterocyclyl is 6 to 14 membered, and more preferably 7 to 10 (for example 7, 8, 9 or 10) membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused heterocyclyl. Representative examples of fused heterocyclyl include, but are not limited to, the following groups:

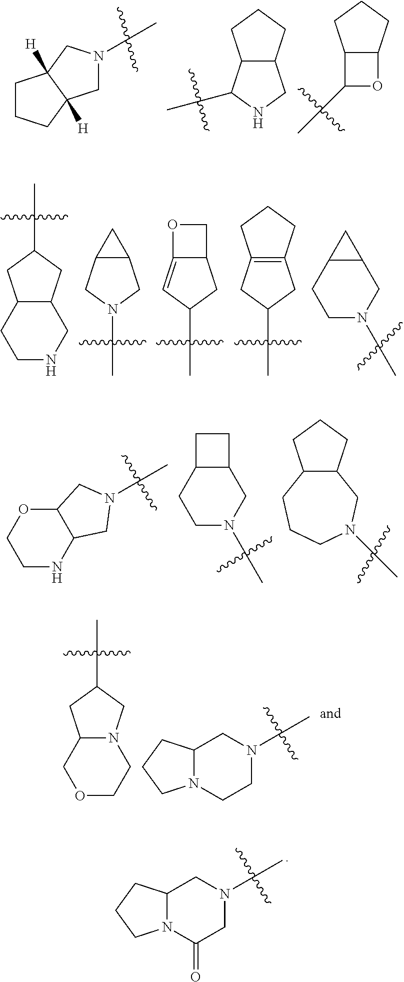

“Bridged Heterocyclyl” refers to a 5 to 14 membered polycyclic heterocyclyl group, wherein every two rings in the system share two disconnected atoms, the rings can have one or more, preferably one to three, double bonds, but have no completely conjugated pi-electron system, and the rings have one or more, preferably one to five, and sometimes more preferably one to three, heteroatoms independently selected from N, O, S, S(O) and S(O)₂ as ring atoms, the remaining ring atoms being C. Preferably a bridged heterocyclyl is 6 to 14 membered, and more preferably 7 to 10 (for example 7, 8, 9 or 10) membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

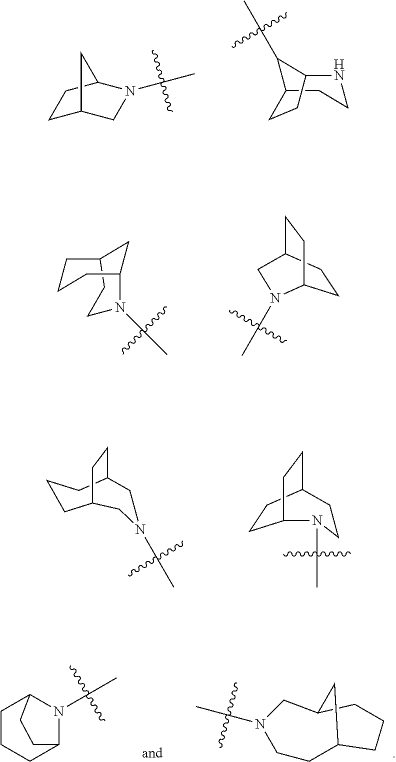

The ring of said heterocyclyl include the heterocyclyl said above which fused to the ring of an aryl, heteroaryl or cycloalkyl, wherein the ring bound to the parent structure is heterocyclyl. Representative examples include, but are not limited to the following groups:

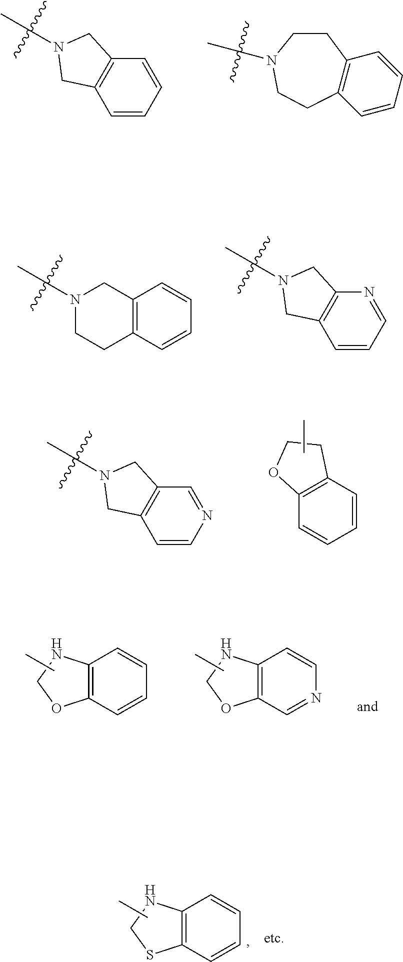

The heterocyclyl is optionally substituted or unsubstituted. When substituted, the substituent group(s) is preferably one or more, preferably one to five, and sometimes more preferably one to three, group(s) independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl include the aryl said above which fused to the ring of heteroaryl, heterocyclyl or cycloalkyl, wherein the ring bound to parent structure is aryl. Representative examples include, but are not limited to, the following groups:

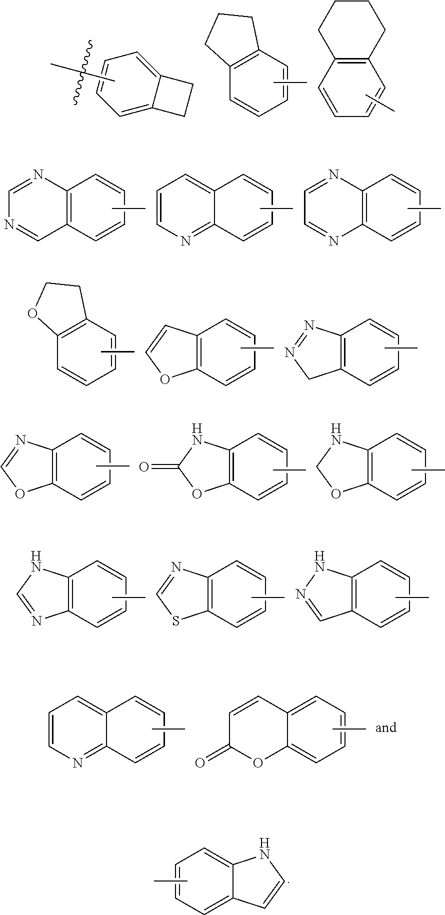

The aryl group can be substituted or unsubstituted. When substituted, the substituent group(s) is preferably one or more, preferably one to five, and sometimes more preferably one to three, groups independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

“Heteroaryl” refers to an aryl system having 1 to 4 (for example 1, 2, 3 or 4) heteroatoms selected from O, S and N as ring atoms and having 5 to 14 annular atoms. Preferably a heteroaryl is 5- to 10- (for example 5, 6, 7, 8, 9 or 10) membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl include the heteroaryl said above which fused with the ring of an aryl, heterocyclyl or cycloalkyl, wherein the ring bound to parent structure is heteroaryl. Representative examples include, but are not limited to, the following groups:

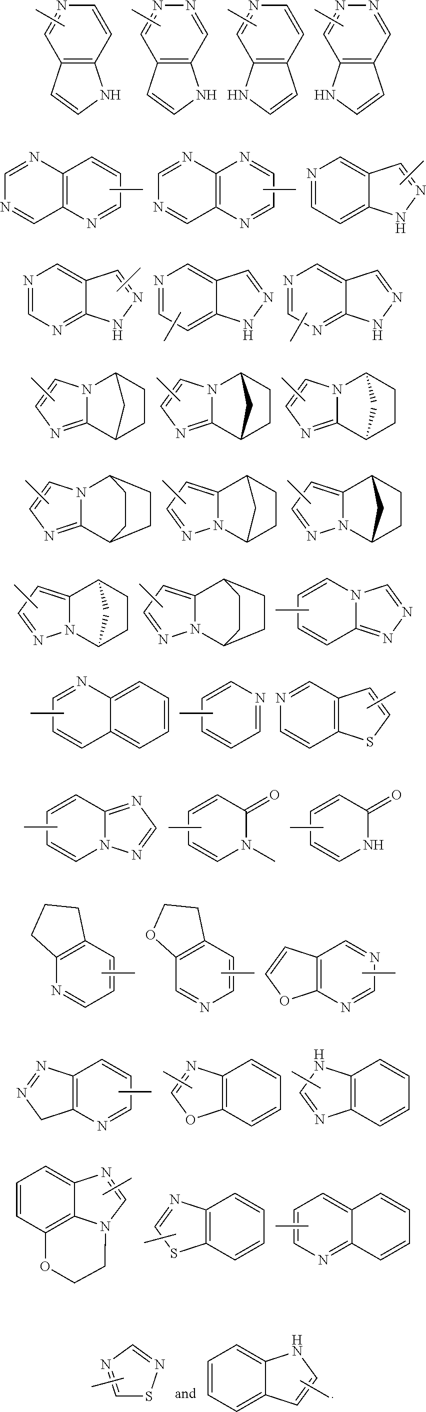

The heteroaryl group can be substituted or unsubstituted. When substituted, the substituent group(s) is preferably one or more, preferably one to five, and sometimes more preferably one to three, groups independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxy can be substituted or unsubstituted. When substituted, the substituent is preferably one or more, preferably one to five, and sometimes more preferably one to three, groups independently selected from alkyl, alkenyl, alkynyl, alkoxy, alkylthio, alkylamino, halogen, thiol, hydroxyl, nitro, cyano, cycloalkyl, heterocyclyl, aryl, heteroaryl, cycloalkoxy, heterocyclyloxy, cycloalkylthio and heterocyclylthio.

The term “amino protecting group” is to keep the amino group unchanged when other parts of the molecule react, and to protect the amino group with a group that is easy to remove. Non-limiting examples include (trimethylsilyl)ethoxymethyl, tetrahydropyranyl, t-butoxycarbonyl, acetyl, benzyl, allyl, p-methoxybenzyl, and the like. These groups may be optionally substituted with 1-3 substituents selected from halogen, alkoxy or nitro. Preferably is tetrahydropyranyl; further preferably is tetrahydropyran-2-yl.

“Bond” refers to a covalent bond using a sign of “—”.

“Hydroxyalkyl” refers to an alkyl group substituted by a hydroxyl group, wherein alkyl is as defined above.

“Hydroxyl” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(═O)OH group.

OTf refers to F

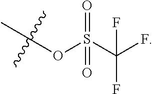 [SMILES: *OS(=O)(=O)C(F)(F)F]

“Alkoxycarbonyl” refers to a —C(═O)O(alkyl) or —C(═O)O (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity.

The compounds of the present disclosure may also contain unnatural proportions of atomic isotopes at one or more of the atoms that constitute such compounds. Unnatural propoliions of an isotope may be defined as ranging from the amount found in nature to an amount consisting of 100/6 of the atom in question. For example, the compounds may incorporate radioactive isotopes, such as for example tritium (³H), iodine-125 (¹²⁵I) or carbon-14 (¹⁴C), or non-radioactive isotopes, such as deuterium (D) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents.

### Synthesis Method of the Compound of the Present Disclosure

In order to complete the purpose of the disclosure, the present disclosure applies, but is not limited to, the following technical solution:

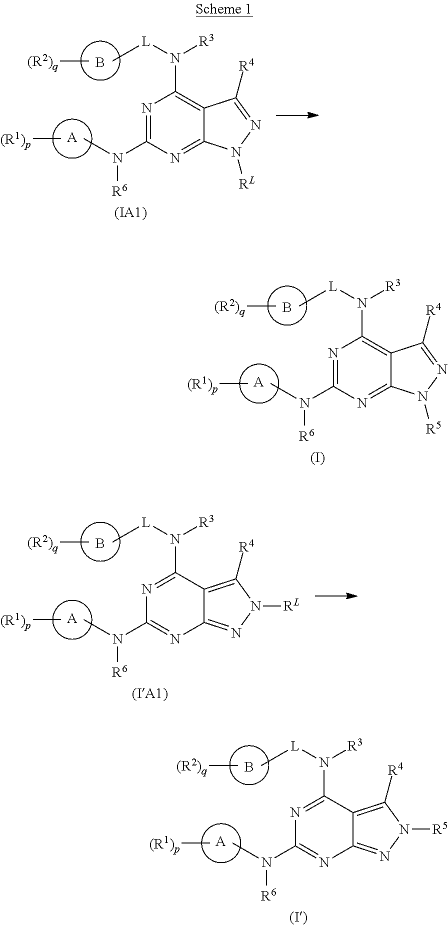

A preparation process of a compound of formula (I) or (I′), or a tautomer, mesomer, racemate, enantiomer, diastereomer, or mixture thereof, or a pharmaceutically acceptable salt thereof, comprising a step of:

the compound of formula (IA1) or (I′A1) remove the group R^{L} in the presence of acid, to obtain the compound of formula (I) or (I′), wherein:

R^{L} is a protecting group; preferably is tetrahydropyranyl;

R⁵ is H;

ring A, ring B, L, R¹ to R⁴, R⁶, p and q are each as defined in formula (I) or (I′).

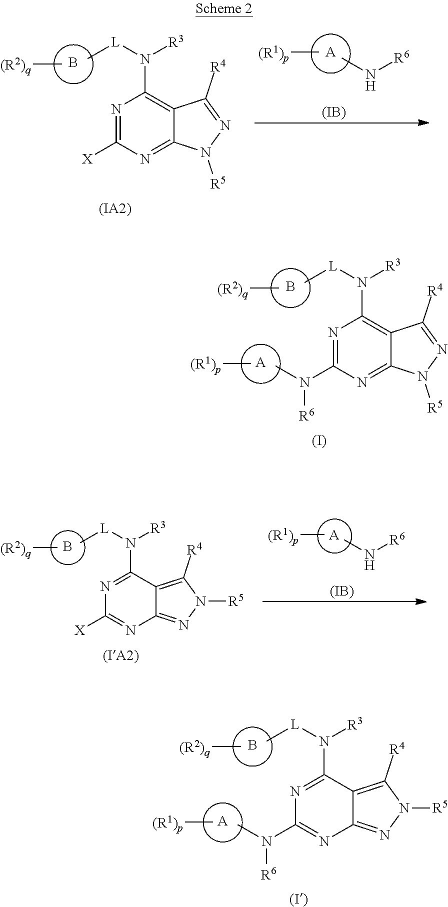

A preparation process of a compound of formula (I) or (I′), or a tautomer, mesomer, racemate, enantiomer, diastereomer, or mixture thereof, or a pharmaceutically acceptable salt thereof, comprising a step of:

formula (IA2) or (I′A2) and formula (IB) or a pharmaceutically acceptable salt thereof perform Buchwald coupling reaction under basic conditions in the presence of a metal catalyst, or perform SnAr reaction in the presence of acid, to obtain the compound of formula (I) or (I′), wherein:

X is halogen;

the pharmaceutically acceptable salt of the compound (IB) preferably is hydrochloride; and

ring A, ring B, L, R¹ to R⁶, p and q are each as defined in formula (I) or (I′).

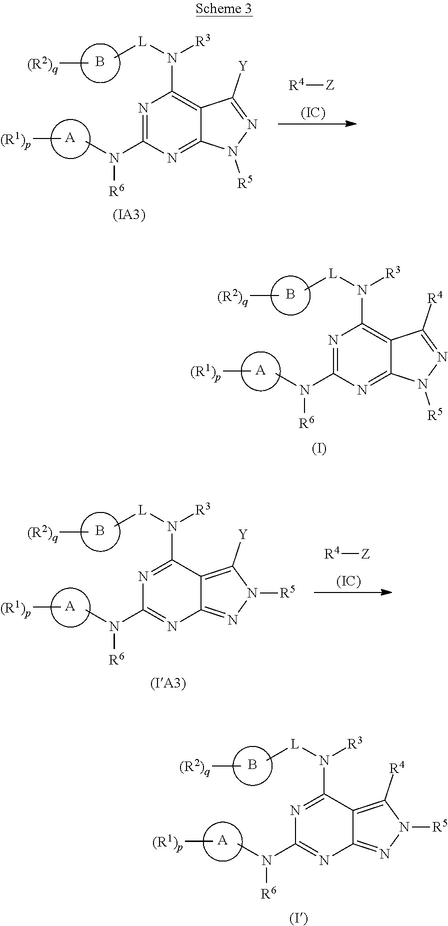

A preparation process of a compound of formula (I) or (I′), or a tautomer, mesomer, racemate, enantiomer, diastereomer, or mixture thereof, or a pharmaceutically acceptable salt thereof, comprising a step of:

formula (IA3) or (I′A3) and formula (IC) perform coupling reaction in the presence of a metal catalyst, optionally under basic conditions, to obtain the compound of formula (I) or (I′), wherein:

Y is halogen or OTf;

Z is selected from

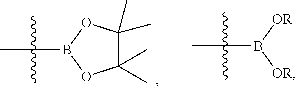

—MgX′, —ZnY′, BF₃, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen; ring A, ring B, L, R¹ to R⁶, R^{a}, R^{b}, p and q are each as defined in formula (I) or (I′).

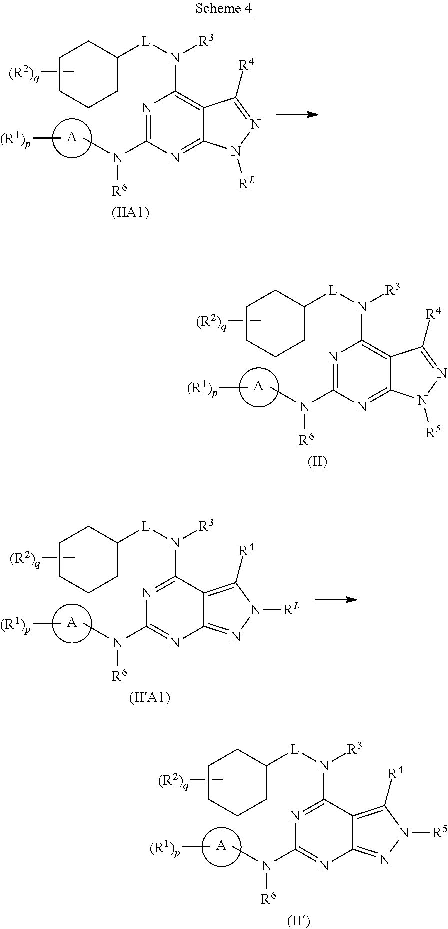

A preparation process of a compound of formula (II) or (II′), or a tautomer, mesomer, racemate, enantiomer, diastereomer, or mixture thereof, or a pharmaceutically acceptable salt thereof, comprising a step of:

the compound of formula (IIA1) or (II′A1) remove the group R^{L} in the presence of acid, to obtain the compound of formula (II) or (II′), wherein:

R^{L} is a protecting group; preferably is tetrahydropyranyl;

R⁵ is H;

ring A, L, R¹ to R⁴, R⁶, p and q are each as defined in formula (II) or (II′).

A preparation process of a compound of formula (II) or (II′), or a tautomer, mesomer, racemate, enantiomer, diastereomer, or mixture thereof, or a pharmaceutically acceptable salt thereof, comprising a step of:

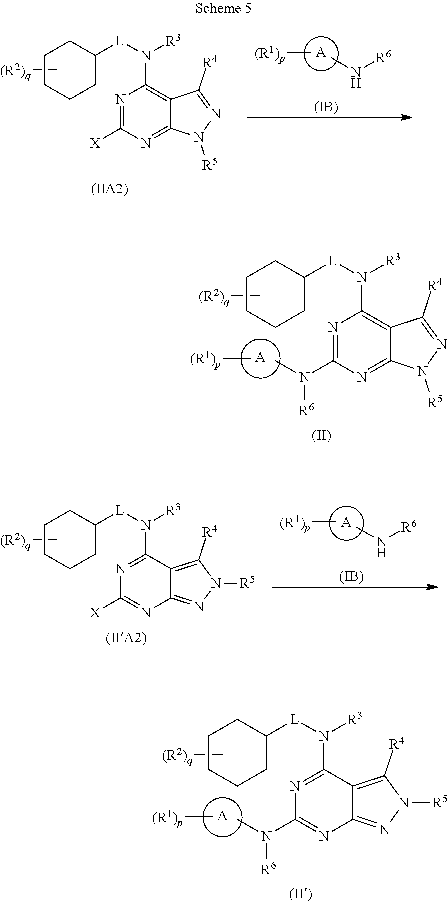

formula (IIA2) or (II′A2) and formula (IB) or a pharmaceutically acceptable salt thereof perform Buchwald coupling reaction under basic conditions in the presence of a metal catalyst, or perform SnAr reaction in the presence of acid, to obtain the compound of formula (II) or (II′), wherein:

X is halogen;

the pharmaceutically acceptable salt of the compound (IB) preferably is hydrochloride; and

ring A, L, R¹ to R⁶, p and q are each as defined in formula (II) or (II′).

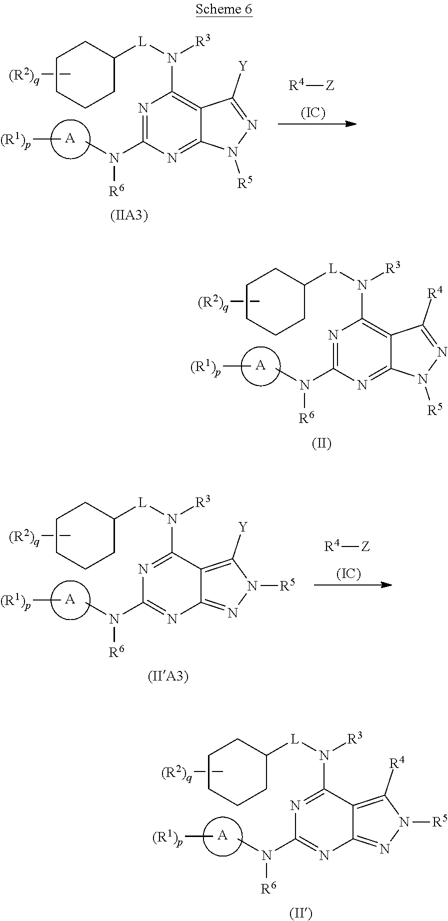

A preparation process of a compound of formula (II) or (II′), or a tautomer, mesomer, racemate, enantiomer, diastereomer, or mixture thereof, or a pharmaceutically acceptable salt thereof, comprising a step of:

formula (IIA3) or (II′A3) and formula (IC) perform Suzuki reaction under basic conditions in the presence of a metal catalyst, to obtain the compound of formula (II) or (II′), wherein:

Y is halogen or OTf;

Z is selected from

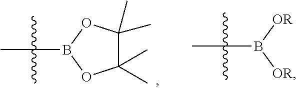

—MgX′, —ZnY′, BF₃—, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen;

ring A, L, R¹ to R⁶, R^{a}, R^{b}, p and q are each as defined in formula (II) or (II′).

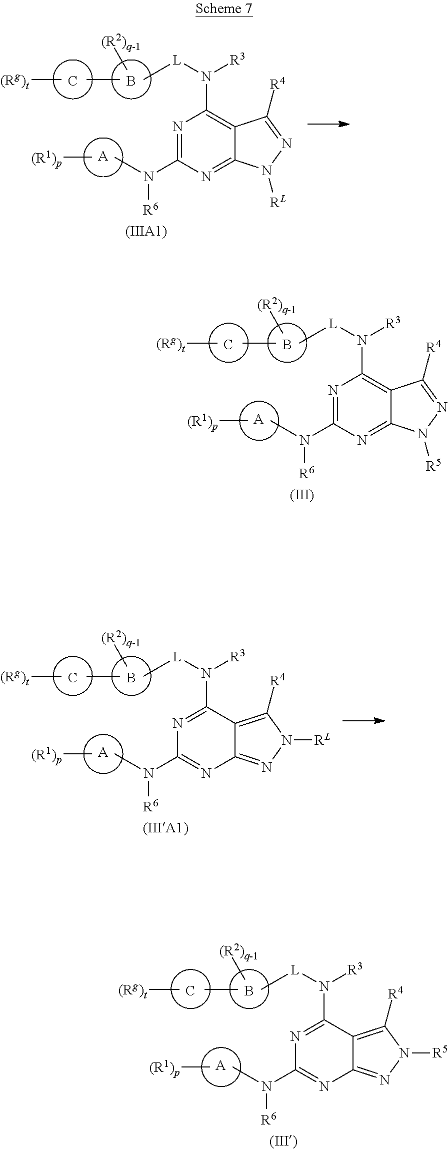

A preparation process of a compound of formula (III) or (III′), or a tautomer, mesomer, racemate, enantiomer, diastereomer, or mixture thereof, or a pharmaceutically acceptable salt thereof, comprising a step of:

the compound of formula (IIIA1) or (III′A1) remove the group R^{L} in the presence of acid, to obtain the compound of formula (III) or (III′), wherein:

R^{L} is a protecting group; preferably is tetrahydropyranyl;

R⁵ is H;

ring A, ring B, ring C, L, R¹ to R⁴, R⁶, R^{g}, t, p and q are each as defined in formula (III) or (III′).

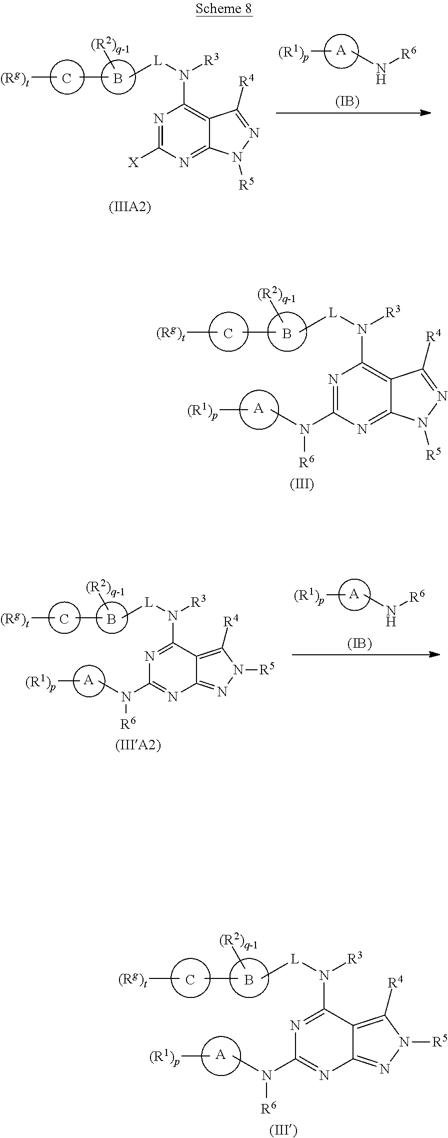

A preparation process of a compound of formula (III) or (III′), or a tautomer, mesomer, racemate, enantiomer, diastereomer, or mixture thereof, or a pharmaceutically acceptable salt thereof, comprising a step of:

formula (IIIA2) or (III′A2) and formula (IB) or a pharmaceutically acceptable salt thereof perform Buchwald coupling reaction under basic conditions in the presence of a metal catalyst, or perform SnAr reaction in the presence of acid, to obtain the compound of formula (III) or (III′), wherein:

X is halogen;

the pharmaceutically acceptable salt of the compound (JIB) preferably is hydrochloride; and ring A, ring B, ring C, L, R¹ to R⁶, R^{g}, t, p and q are each as defined in formula (III) or (III′).

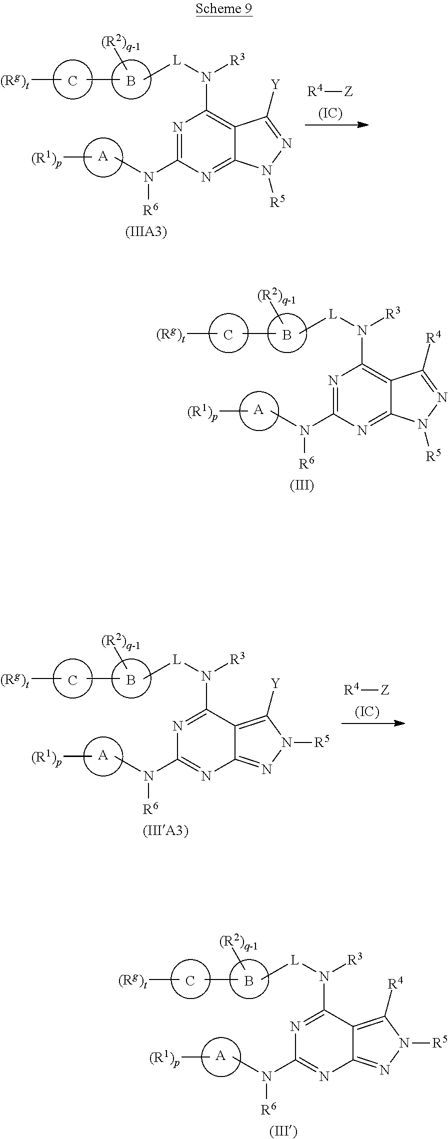

A preparation process of a compound of formula (III) or (III′), or a tautomer, mesomer, racemate, enantiomer, diastereomer, or mixture thereof, or a pharmaceutically acceptable salt thereof, comprising a step of:

formula (IIIA3) or (III′A3) and formula (IC) perform Suzuki reaction under basic conditions in the presence of a metal catalyst, to obtain the compound of formula (III) or (III′), wherein:

Y is halogen or OTf;

Z is selected from

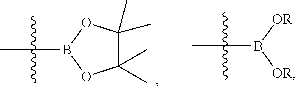

—MgX′, —ZnY′, BF₃—, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen;

ring A, ring B, ring C, L, R¹ to R⁶, R^{a}, R^{b}, R^{g}, t, p and q are each as defined in formula (III) or (III′).

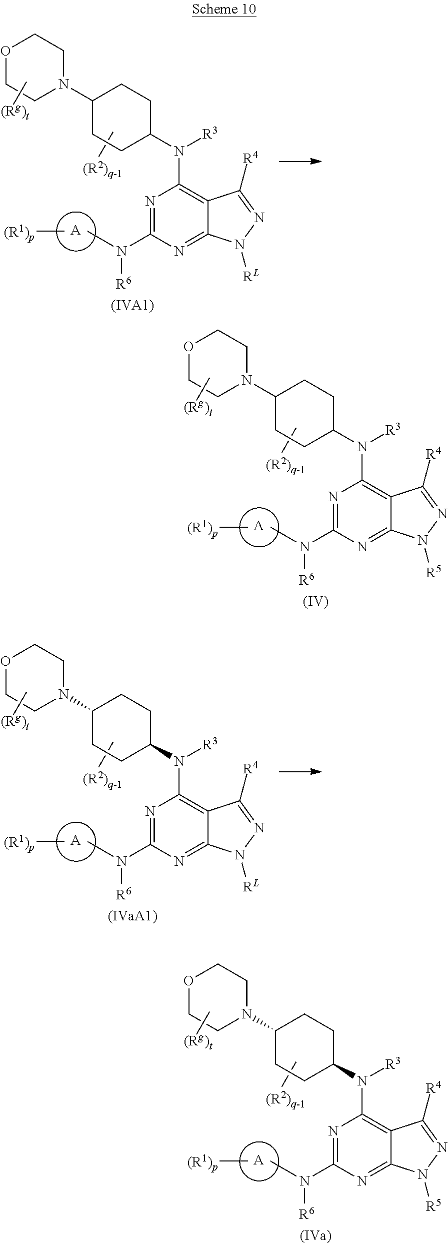

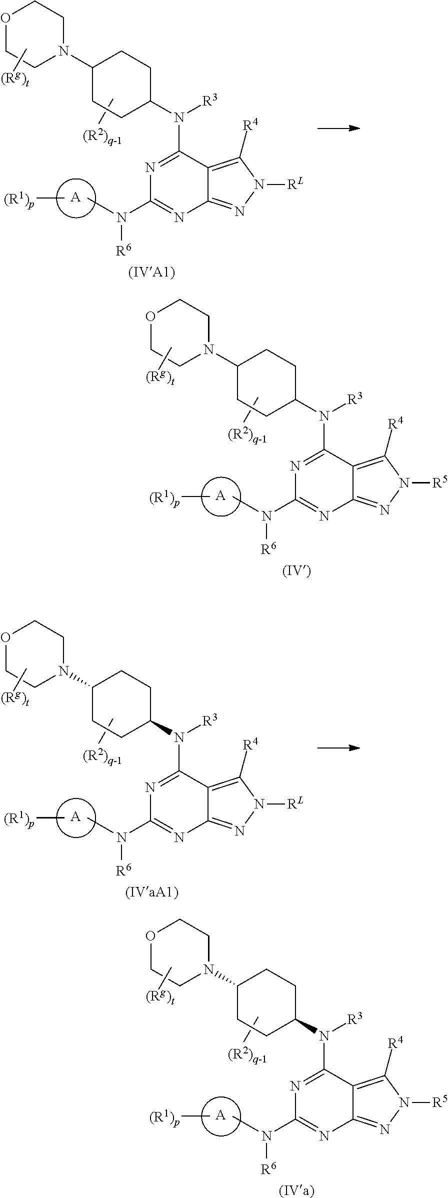

A preparation process of a compound of formula (IV), (IV′), (IVa) or (IV′a), or a tautomer, mesomer, racemate, enantiomer, diastereomer, or mixture thereof, or a pharmaceutically acceptable salt thereof, comprising a step of:

the compound of formula (IVA1), (IV′A1), (IVaA1) or (IV′aA1) remove the group R^{L} in the presence of acid, to obtain the compound of Formula (IV), (IV′), (IVa) or (IV′a), wherein:

R^{L} is a protecting group; preferably is tetrahydropyranyl;

R⁵ is H;

ring A, R¹ to R⁴, R⁶, R^{g}, t, p and q are e in formula (IV) or (IV′).

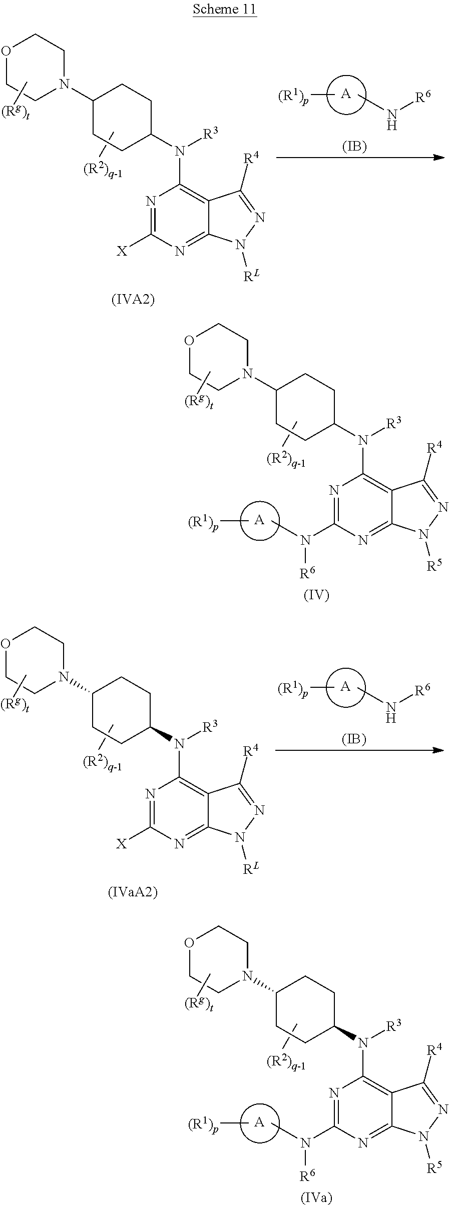

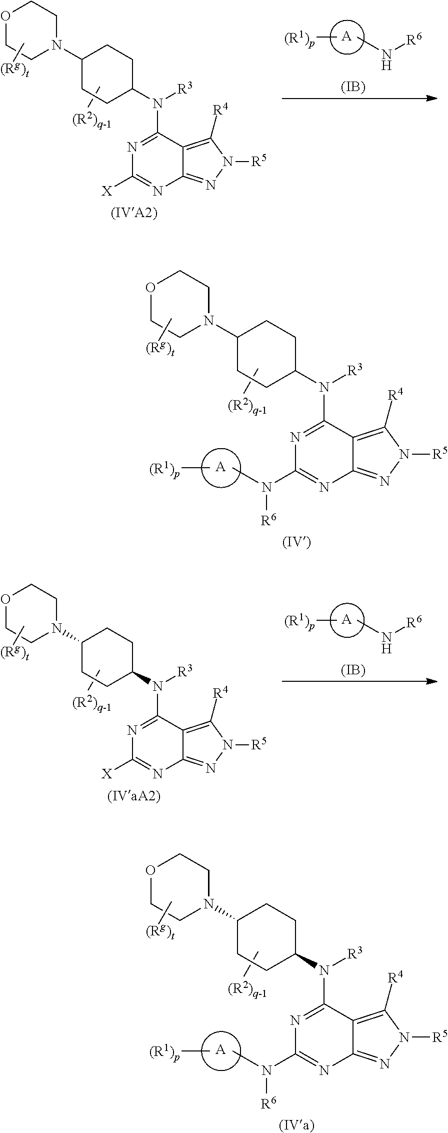

A preparation process of a compound of formula (IV), (IV′), (IVa) or (IV′a), or a tautomer, mesomer, racemate, enantiomer, diastereomer, or mixture thereof, or a pharmaceutically acceptable salt thereof, comprising a step of:

formula (IVA2), (IV′A2), (IVaA2) or (IV′aA2) and formula (IB) or a pharmaceutically acceptable salt thereof perform Buchwald coupling reaction under basic conditions in the presence of a metal catalyst, or perform SnAr reaction in the presence of acid, to obtain the compound of formula (IV), (IV′), (IVa) or (IV′a), wherein:

X is halogen;

the pharmaceutically acceptable salt of the compound (IB) preferably is hydrochloride; and

ring A, R¹-R⁶, R^{g}, t, p and q are each as defined in formula (IV) or (IV′).

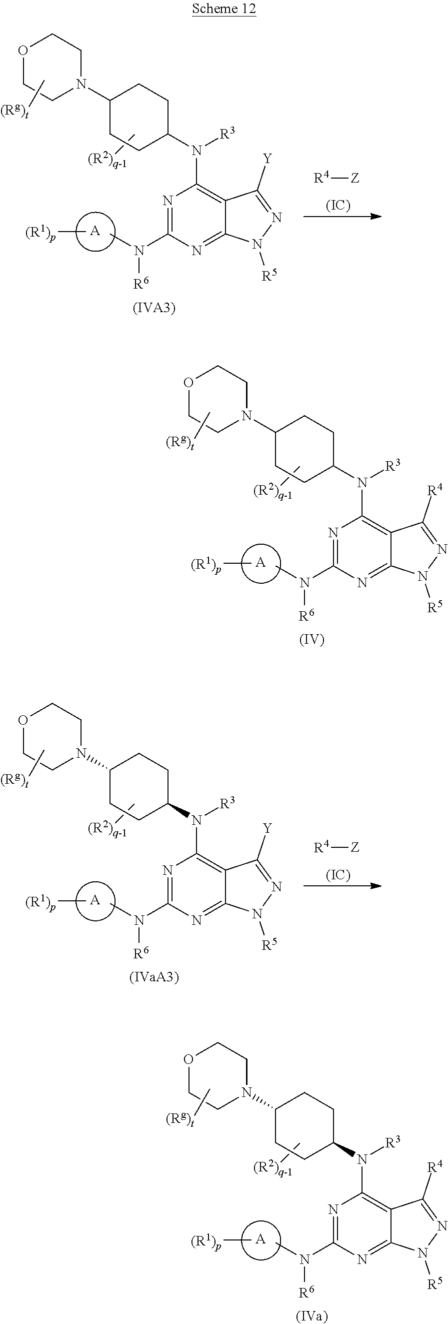

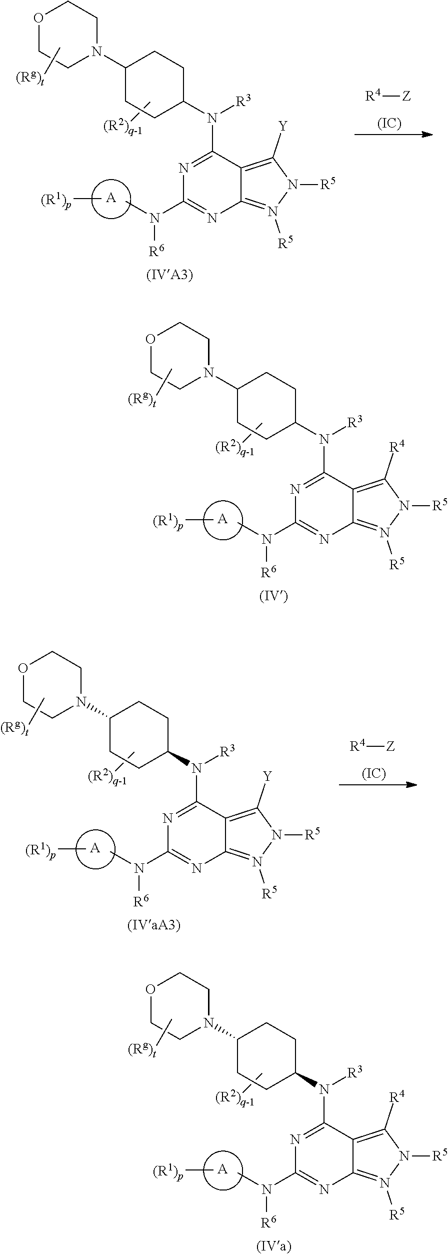

A preparation process of a compound of formula (IV), (IV′), (IVa) or (IV′a), or a tautomer, mesomer, racemate, enantiomer, diastereomer, or mixture thereof, or a pharmaceutically acceptable salt thereof, comprising a step of:

formula (IVA3), (IV′A3), (IVaA3) or (IV′aA3) and formula (IC) perform Suzuki reaction under basic conditions in the presence of a metal catalyst, to obtain the compound of formula (IV), (IV′), (IVa) or (IV′a), wherein:

Y is halogen or OTf;

Z is selected from

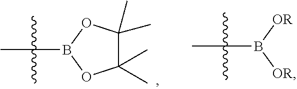

—MgX′, —ZnY′, BF₃—, —Sn(R)₃, —Si(R)₃ and hydrogen;

R is hydrogen or alkyl;

X′ and Y′ are halogen;

provided that only when R⁴ is —NR^{a}R^{b}, alkoxy or haloalkoxy, Z is hydrogen;

ring A, R¹-R⁶, R^{a}, R^{b}, R^{g}, t, p and q are each as defined in formula (IV) or (IV′).

The coupling reaction is selected from Suzuki coupling, Negishi coupling, Stille coupling, Molander coupling, Kumada coupling, Hiyama coupling and Buchwald coupling reaction; preferably is Suzuki coupling reaction.

The acid include, but are not limited to, acetic acid, pyridine hydrobromide, trifluoroacetic acid, formic acid, hydrochloric acid, sulfuric acid, p-toluenesulfonic acid, p-toluenesulfonic acid monohydrate and methanesulfonic acid; preferably hydrochloric acid or p-toluenesulfonic acid monohydrate.

The reagents that provide basic conditions includes organic bases and inorganic bases, wherein the organic base includes, but is not limited to, triethylamine, N,N-disopropylethylamine, n-butyllithium, lithium diisopropylamide, potassium acetate, sodium tert-butoxide, N-(3-Dimethylaminopropyl)-N′-ethylcarbodiimide hydrochloride (EDCI), potassium bis(trimethylsilyl)amide (KHMDS) and potassium tert-butoxide, and wherein the inorganic base includes, but is not limited to, magnesium chloride, sodium hydride, potassium phosphate, sodium carbonate, potassium carbonate, cesium carbonate and sodium hydroxide; preferably potassium carbonate, cesium carbonate and KHMDS.

The metal catalyst includes, but is not limited to, tris(dibenzylideneacetone)dipalladium (Pd₂(dba)₃), chloro[4-(di-tert-butylphosphino)-N,N-dimethylaniline-2-(2′-aminobiphenyl)]palladium(II) (APhos-Pd-G2), XantPhos, tetrakis(triphenylphosphine)palladium, palladium dichloride, palladium acetate, bis[1,2-bis(diphenylphosphino)ethane]palladium(0)(Pd(dppe)₂), [1,1′-bis(diphenylphosphino)ferrocene]dichloropalladium, 1,1′-bis(diphenylphosphino) ferrocene palladium dichloride or tris(dibenzylideneacetone)dipalladium; preferably Pd₂(dba)₃ or APhos-Pd-G2.

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N, N-dimethylformamide and the mixture thereof.

### Methods of Preparation

### General Synthetic Schemes

The compounds of the present invention can be synthesized using the methods described below, together with synthetic methods known in the art of synthetic organic chemistry, or variations thereon as appreciated by those skilled in the art. Preferred methods include, but are not limited to, those described below. The reactions and techniques described in this section are performed in solvents appropriate to the reagents and materials employed and are suitable for the transformations being affected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. {Protective Groups in Organic Synthesis, Third Edition, Wiley and Sons (1999)). Compounds of the Formula (I) or (I′) can be prepared according to the methods outlined in the following schemes.

In scheme A, 4,6-dichloro-1H-pyrazolo[3,4-d]pyrimidine is converted to its THP protection product 2. An SnAr reaction of intermediate 2 with amine in the presence of amine such as N-ethyl-N-isopropylpropan-2-amine or cesium carbonate could produce intermediate 3. A coupling reaction could install the second amino-group to give intermediate 4, which is then de-protected to give compound 5.

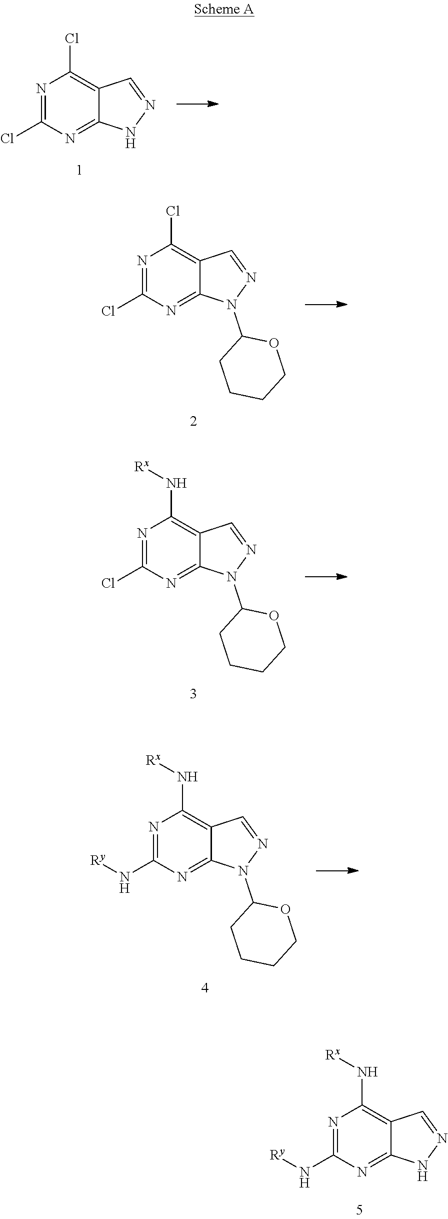

In Scheme B, the intermediate 7 can be prepared from chloride 6 by a SnAr reaction in the presence of base such as N-ethyl-N-isopropylpropan-2-amine or cesium carbonate. An acid-catalyzed SnAr reaction with p-toluenesulfonic acid monohydrate would furnish intermediate 8. A Suzuki coupling reaction with a boronic acid can afford compound 9.

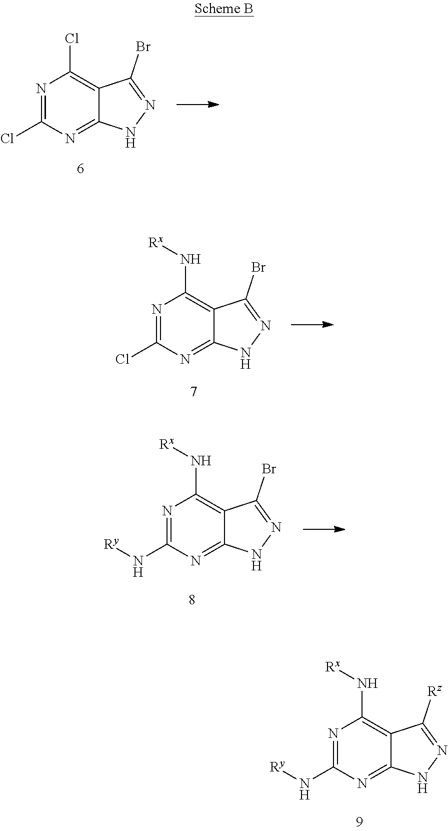

In Scheme C, the compound 10 or 13 is purchased commercially, or is obtained by introducing compound 1 (4,6-dichloro-1H-pyrazolo[3,4-d]pyrimidine) into R⁵ through alkylation, acylation or sulfonylation reaction. Then undergoes two SnAr reactions to obtain compound 12 or 15.

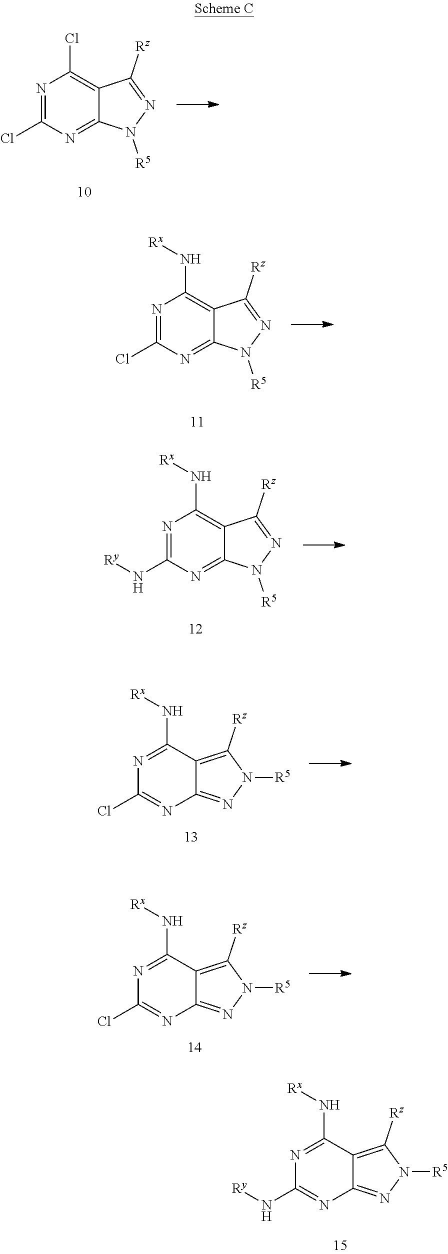

### Experimental Procedures and Working Examples

The following illustrate the synthesis of various compounds of the present invention. Additional compounds within the scope of this invention may be prepared using the methods illustrated in these Examples, either alone or in combination with techniques generally known in the art. It will be understood that the intermediate compounds of the invention depicted above are not limited to the particular enantiomer shown, but also include all stereoisomers and mixtures thereof. It will also be understood that compounds of Formula (I) or (I′) can include intermediates of compounds of Formula (I) or (I′).

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, δ) referenced to residual peaks from the deuterated solvents employed.

For syntheses referencing procedures in other Examples or Methods, reaction conditions (length of reaction and temperature) may vary. In general, reactions were followed by thin layer chromatography and/or liquid chromatography-mass spectrometry, and subjected to work-up when appropriate. It will be recognized by one skilled in the art that purifications may vary between experiments: in general, sorbents, solvents and the solvent ratios used for eluants/gradients were chosen to provide appropriate Rf s or retention times. It will also be recognized by one skilled in the art that HPLC purifications may be effected in a variety of ways, including the use of normal stationary phases, reverse stationary phases, chiral stationary phases, and supercritical eluants. The appropriate choices of conditions for chromatographic and HPLC purifications will be discerned by one skilled in the art. The following Preparations describe the preparation of certain intermediates used in the Methods and Examples that follow.

The following Preparations, Methods and Examples are intended to illustrate particular embodiments of the invention and preparations thereto and are not intended to limit the specification, including the claims, in any manner. Unless noted otherwise, all reactants were obtained commercially.

Unless indicated otherwise, the following abbreviations have the indicated meanings:
APCI—atmospheric pressure chemical ionization
br.—broad peaks
° C.—degree Celsius
CDCL3—deuterated chloroform
CD₃OD—deuterated methanol
d—doublet peak
dd—double doublet peak
D₂O—deuterium oxide
dmso-ds—perdeuterated dimethyl sulfoxide
dt—double triplet peak
g—gram(s)
H (e.g., 1H, 2H)—hydrogen(s)
hr—hour(s)
LC—liquid chromatography
m—multiplet
M—molarity
mg—milligram(s)
MHz—megahertz
min—minute(s)
mL—milliliter(s)
mmol—millimole(s)
mp—melting point
MS—mass spectrum
NMR—nuclear magnetic resonance
pH—negative logarithm of hydronium ion concentration
psi—pounds per square inch
q—quartet peak
s—singlet peak
t—triplet peak
td—triple doublet peak
μL—microliter

Unless indicated otherwise, the following chemical formulas and acronyms have the indicated meanings:
AcOH—glacial acetic acid
BF₃-Et₂O—boron trifluoride etherate
BHT—2,6-dit-t-butyl-4-methylphenol
CHCl₃—chloroform
DAST—(diethylamino)sulfur trifluoride
DCM—dichloromethane
DMAP—(4-dimethylamino)pyridine
DMF—dimethylformamide
DMSO—dimethylsulfoxide
EDCI—N-(3-Dimethylaminopropyl)-N′-ethylcarbodiirnide hydrochloride
Et₃N—triethylamine
EtOAc—ethyl acetate
EtOH—ethanol
HCl—hydrochloric acid
HNO₃—nitric acid
H₂SO₄—sulfuric acid
H₃PO₄—phosphoric acid
LDA—lithium diisopropylamide
MeCN—acetonitrile
MeOH—methanol
Mg₂SO₄—anhydrous magnesium sulfate
K₂CO₃—potassium carbonate
K3PO₄—anhydrous tribasic potassium phosphate
KOH—potassium hydroxide
MTBE—methyl t-butyl ether
Na₂CO₃—sodium carbonate
Na₂SO₄—anhydrous sodium sulfate
NaBH₄—sodium borohydride
NaHCO₃—sodium bicarbonate
NaOH—sodium hydroxide
NFSI—N-fluoro(bisbenzenesulfonyl)imide, CAS 133745-75-2
NH₄Cl—ammonium chloride
POCl₃—phosphorus oxychloride
TFA—trifluoroacetic acid
THF—tetrahydrofuran
TMSCI—chlorotrimethylsilane

### Preparations

### Example 1: 3-bromo-N⁴-((1r,4r)-4-morpholinocyclohexyl)-N⁶-(1-(tetrahydro-2H-pyran-4-yl)-1H-pyrazol-4-yl)-1H-pyrazolo[3,4-d]pyrimidine-4,6-diamine 1

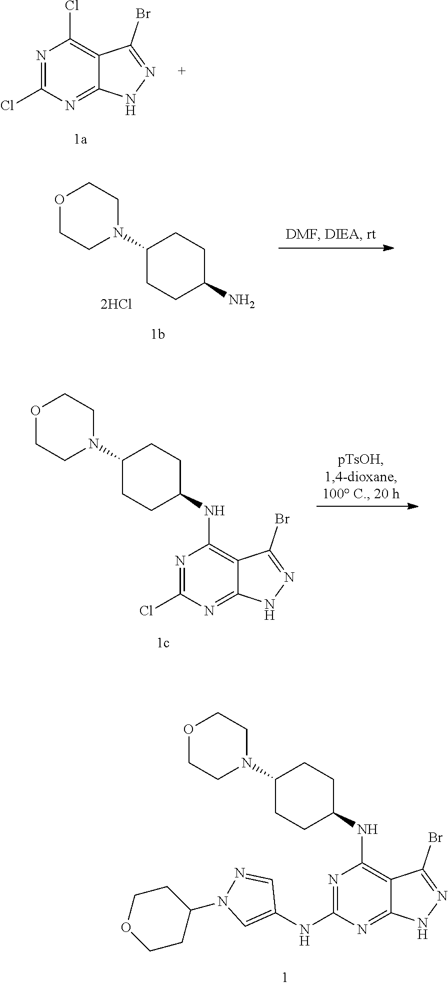 [SMILES: Clc1nc(Cl)c2c(Br)n[nH]c2n1 ; N[C@H]1CC[C@H](N2CCOCC2)CC1 ; Clc1nc(N[C@H]2CC[C@H](N3CCOCC3)CC2)c2c(Br)n[nH]c2n1 ; Brc1n[nH]c2nc(Nc3cnn(C4CCOCC4)c3)nc(N[C@H]3CC[C@H](N4CCOCC4)CC3)c12]

### Step 1. Synthesis of 3-bromo-6-chloro-N-((1r,4r)-4-morpholinocyclohexyl)-1H-pyrazolo[3,4-d]pyrimidin-4-amine 1c

To a solution of trans-(1r,4r)-4-morpholinocyclohexan-1-amine dihydrochloride 1b (565.8 mg, 2.2 mol, Combi-Blocks) in anhydrous DMF (10 mL), was added N-ethyl-N-isopropylpropan-2-amine (DIEA, 426 mg, 3.3 mmol), followed by 3-bromo-4,6-dichloro-1H-pyrazolo[3,4-d]pyrimidine 1a (535.8 mg, 2 mmol, Ambeed). The resulting mixture was stirred at rt for 20 h. The mixture was diluted with 20 mL of ethyl acetate and 10 mL of water. The organic layer was separated, and the aqueous layer was extracted with ethyl acetate (10 mL). The combined organic layers were washed with water and brine, dried over sodium sulfate, filtered and concentrated. The crude product was purified by flash chromatography (0%4-10% dichloromethane in methanol) to give 145.2 mg of 3-bromo-6-chloro-N-((1r,4r)-4-morpholinocyclohexyl)-1H-pyrazolo[3,4-d]pyrimidin-4-amine 1c (0.35 mmol, 17.5% yield).

### Step 2. Synthesis of 3-bromo-N⁴-((1r,4r)-4-morpholinocyclohexyl)-N⁶-(1-(tetrahydro-2H-pyran-4-yl)-1H-pyrazol-4-yl)-1H-pyrazolo[3,4-d]pyrimidine-4,6-diamine 1

To a solution of 3-bromo-6-chloro-N-((1r,4r)-4-morpholinocyclohexyl)-1H-pyrazolo[3,4-d]pyrimidin-4-amine 1c (145.2 mg, 0.35 mmol) in 1,4-dioxane (15 mL), was added 1-(tetrahydro-2H-pyran-4-yl)-1H-pyrazol-4-amine (70.2 mg, 0.42 mmol, Combi-Blocks), followed by p-toluenesulfonic acid monohydrate (80 mg, 0.42 mmol). The resulting mixture was heated at 100° C. for 20 h. After being cooled to rt, the mixture was diluted with 20 mL of ethyl acetate and 10 mL of saturated sodium carbonate aqueous solution. The organic layer was separated, and the aqueous layer was extracted with ethyl acetate (10 mL). The combined organic layers were washed with brine, dried over sodium sulfate, filtered and concentrated. The crude product was purified by flash chromatography (0%-10% dichloromethane in methanol) to give 86.5 mg of 3-bromo-N⁴-((1r,4r)-4-morpholinocyclohexyl)-N⁶-(1-(tetrahydro-2H-pyran-4-yl)-1H-pyrazol-4-yl)-1H-pyrazolo[3,4-d]pyrimidine-4,6-diamine 1 (0.16 mmol, 45% yield). MS m/z (ESI): 546 [M+1]. ¹H NMR (400 MHz, DMSO-d₆) δ 13.00 (s, 1H), 9.09 (s, 1H), 7.96-7.89 (m, 1H), 7.53-7.50 (m, 1H), 6.10-5.92 (m, 1H), 4.36-4.30 (m, 1H), 4.05-3.95 (m, 4H), 3.58 (t, 4H), 3.46 (t, 2H), 2.31-2.25 (m, 2H), 2.11 (m, 2H), 1.99-1.90 (m, 7H), 1.47-1.37 (m, 5H).

### Example 2: N⁴-((1r,4r)-4-morpholinocyclohexyl)-3-(pyridin-3-yl)-N⁶-(1-(tetrahydro-2H-pyran-4-yl)-1H-pyrazol-4-yl)-1H-pyrazolo[3,4-d]pyrimidine-4,6-diamine 2

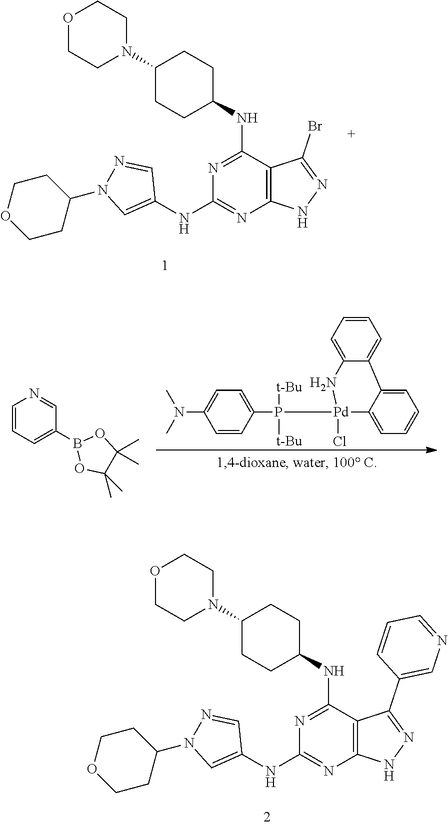

To a solution of 3-bromo-N⁴-((1r,4r)-4-morpholinocyclohexyl)-N⁶-(1-(tetrahydro-2H-pyran-4-yl)-1H-pyrazol-4-yl)-1H-pyrazolo[3,4-d]pyrimidine-4,6-diamine 1 (20 mg, 36.6 μmol) in 1,4-dioxane (0.5 mL) and water (0.2 mL), was added 3-(4,4,5,5-tetramethyl-1,3,2-dioxaborolan-2-yl)pyridine (11.3 mg, 55 μmol, Combi-Blocks), followed by potassium carbonate (15.2 mg, 0.11 mmol) and chloro[4-(di-tert-butylphosphino)-N,N-dimethylaniline-2-(2′-aminobiphenyl)]palladium(II) (APhos-Pd-G2, 1.1 mg, 1.8 μmol, Aldrich). The resulting mixture was bubbled with nitrogen for 5 min then sealed and heated at 100° C. for 20 h. After being cooled to rt, the mixture was diluted with ethyl acetate (2 mL) and water (1 mL). The organic layer was separated, and the aqueous layer was extracted with ethyl acetate (2 mL). The combined organic layers were washed with brine, dried over sodium sulfate, filtered and concentrated. The residue was purified by chromatography (0-10% methanol in dichloromethane) to give 11.4 mg of N⁴-((1r,4r)-4-morpholinocyclohexyl)-3-(pyridin-3-yl)-N⁶-(1-(tetrahydro-2H-pyran-4-yl)-1H-pyrazol-4-yl)-1H-pyrazolo[3,4-d]pyrimidine-4,6-diamine 2 (21 μmol, 57% yield). MS m/z (ESI): 545 [M+1]. ¹H NMR (400 MHz, Methanol-d₄) δ 8.87 (s, 1H), 8.67 (d, 1H), 8.15 (d, 1H), 8.06 (s, 1H), 7.68 (s, 1H), 7.64-7.61 (m, 2H), 4.61 (s, 1H), 4.42-4.35 (m, 1H), 4.11-4.08 (m, 3H), 3.73 (t, 4H), 3.64-3.57 (m, 2H), 2.65 (m, 4H), 2.33-2.23 (m, 3H), 2.16-2.03 (m, 6H), 1.46-1.26 (m, 5H).

### Example 3: N⁴-((1r,4r)-4-morpholinocyclohexyl)-3-(1H-pyrazol-4-yl)-N⁶-(1-(tetrahydro-2H-pyran-4-yl)-1H-pyrazol-4-yl)-1H-pyrazolo[3,4-d]pyrimidine-4,6-diamine 3

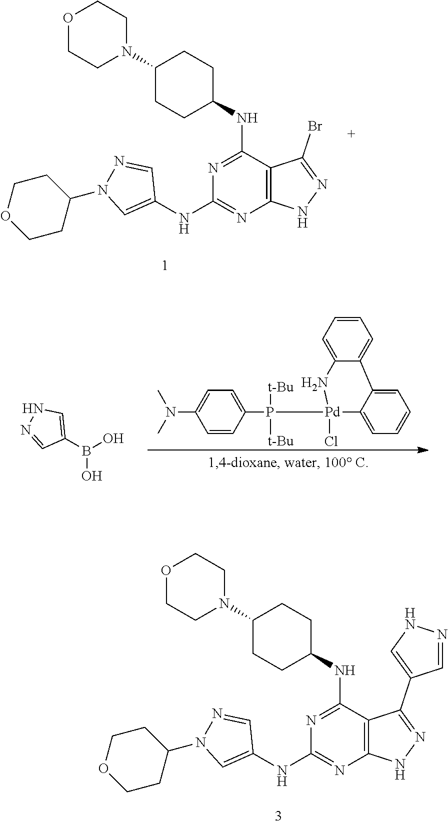

To a solution of 3-bromo-N⁴-((1r,4r)-4-morpholinocyclohexyl)-N⁶-(1-(tetrahydro-2H-pyran-4-yl)-1H-pyrazol-4-yl)-1H-pyrazolo[3,4-d]pyrimidine-4,6-diamine 1 (20 mg, 36.6 μmol) in 1,4-dioxane (0.5 mL) and water (0.2 mL), was added (I H-pyrazol-4-yl)boronic acid (13 mg, 110 μmol, Combi-Blocks), followed by potassium carbonate (15.2 mg, 0.11 mmol) and Chloro[4-(di-tert-butylphosphino)-N,N-dimethylaniline-2-(2′-aminobiphenyl)]palladium(II) (APhos-Pd-G2, 1.1 mg, 1.8 μmol, Aldrich). The resulting mixture was bubbled with nitrogen for 5 min then sealed and heated at 100° C. for 20 h. After being cooled to rt, the mixture was diluted with ethyl acetate (2 mL) and water (1 mL). The organic layer was separated, and the aqueous layer was extracted with ethyl acetate (2 mL). The combined organic layers were washed with brine, dried over sodium sulfate, filtered and concentrated. The residue was purified by chromatography (0-10% methanol in dichloromethane) to give 3.2 mg of N⁴-((1r,4r)-4-morpholinocyclohexyl)-3-(1H-pyrazol-4-yl)-A*-(1-(tetrahydro-2H-pyran-4-yl)-1H-pyrazol-4-yl)-1H-pyrazolo[3,4-d]pyrimidine-4,6-diamine 3 (6 μmol, 16% yield). MS m/z (ESI). 534 [M+1].

### Example 4: N⁶-(3-methylisothiazol-5-yl)-N⁴-((1r,4r)-4-morpholinocyclohexyl)-1-(tetrahydro-2H-pyran-2-yl)-1H-pyrazolo[3,4-d]pyrimidine-4,6-diamine 4

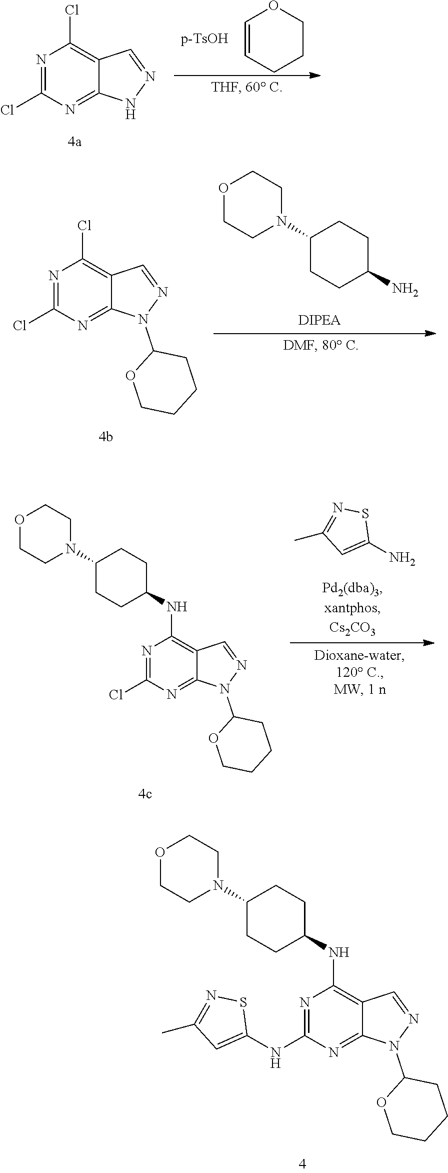

### Step 1. Synthesis of 4,6-dichloro-1-(tetrahydro-2H-pyran-2-yl)-1H-pyrazolo[3,4-d]pyrimidine 4b

To a stirred solution of 4,6-dichloro-1H-pyrazolo[3,4-d]pyrimidine 4a (567 mg, 3 mmol, Combi-Blocks) in THF (10 mL, Aldrich) was added 3,4-dihydro-2H-pyran (410 uL, 4.5 mmol, Combi-Blocks) and P-TsOH (57 mg, 0.3 mmol, Aldrich) at room temperature. The mixture was heated at 60° C. for 2 h and allowed to cool to room temperature. The solvent was removed, and the residue was redissolved in ethyl acetate and washed with sodium bicarbonate, dried over sodium sulfate, filtered and concentrated to afford the title compound 4b (816 mg, 100%). MS m/z (ESI): 273 [M+1].

### Step 2. Synthesis of 6-chloro-N-((1r,4r)-4-morpholinocyclohexyl)-1-(tetrahydro-2H-pyran-2-yl)-1H-pyrazolo[3,4-d]pyrimidin-4-amine 4c

To a stirred solution of 4,6-dichloro-1-(tetrahydro-2H-pyran-2-yl)-1H-pyrazolo[3,4-d]pyrimidine 4b (475 mg, 1.75 mmol) in DMF (5 mL, Aldrich) was added DIPEA (1 mL, 5.77 mmol, Aldrich) and (1r,4r)-4-morpholinocyclohexan-1-amine (494 mg, 1.92 mmol, Combi-blocks). The mixture was heated at 90° C. overnight. The solvent was removed, and the residue was purified by chromatography to afford the title compound 4c (660 mg, 90%). MS m/z (ESI): 421 [M+1].

### Step 3. Synthesis of N⁶-(3-methylisothiazol-5-yl)-N⁴-((1r,4r)-4-morpholinocyclohexyl)-1-(tetrahydro-2H-pyran-2-yl)-1H-pyrazolo[3,4-d]pyrimidine-4,6-diamine 4

A mixture of 6-chloro-N-((1r,4r)-4-morpholinocyclohexyl)-1-(tetrahydro-2H-pyran-2-yl)-1H-pyrazolo[3,4-d]pyrimidin-4-amine 4c (124 mg, 0.29 mmol), 3-methylisothiazol-5-amine hydrogen chloride salt (53 mg, 0.35 mmol, Ambeed), Pd₂dba₃ (27 mg, 0.029 mmol, Aldrich), xantphos (34 mg, 0.059 mmol, Aldrich) and Cs₂CO₃ (288 mg, 0.885 mmol, Aldrich) in dioxane (3 mL, Aldrich) and water (0.3 mL) were heated at 120° C. for 1 h under MW. The solvent was removed, and the residue was purified by chromatography to afford the title compound 4 (102 mg, 70%). MS m/z (ESI): 499 [M+1]. ¹H NMR (400 MHz, Methanol-d₄): δ 8.0 (s, 1H), 6.6 (s, 1H), 4.4-4.0 (m, 2H), 3.9-3.6 (m, 5H), 2.8-2.5 (m, 5H), 2.4 (s, 3H), 2.3-2.0 (m, 6H), 1.9-1.3 (m, 9H).

### Example 5: N⁴-((1r,4r)-4-morpholinocyclohexyl)-3-phenyl-N⁶-(1-(tetrahydro-2H-pyran-4-yl)-1H-pyrazol-4-yl)-1H-pyrazolo[3,4-d]pyrimidine-4,6-diamine 5

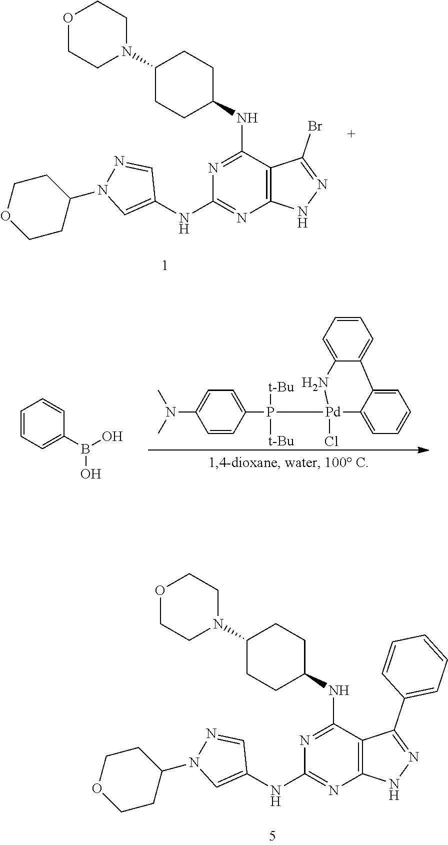

To a solution of 3-bromo-N⁴-((1r,4r)-4-morpholinocyclohexyl)-N⁶-(1-(tetrahydro-2H-pyran-4-yl)-1H-pyrazol-4-yl)-1H-pyrazolo[3,4-d]pyrimidine-4,6-diamine 1 (20 mg, 36.6 μmol) in 1,4-dioxane (0.5 mL) and water (0.2 mL), was added phenyl boronic acid (6.7 mg, 55 μmol, Combi-Blocks), followed by potassium carbonate (15.2 mg, 0.11 mmol) and Chloro[4-(di-tert-butylphosphino)-N,N-dimethylaniline-2-(2′-aminobiphenyl)]palladium(II) (APhos-Pd-G2, 1.1 mg, 1.8 μmol, Aldrich). The resulting mixture was bubbled with nitrogen for 5 min then sealed and heated at 100° C. for 20 h. After being cooled to rt, the mixture was diluted with ethyl acetate (2 mL) and water (1 mL). The organic layer was separated, and the aqueous layer was extracted with ethyl acetate (2 mL). The combined organic layers were washed with brine, dried over sodium sulfate, filtered and concentrated. The residue was purified by chromatography (0-10% methanol in dichloromethane) to give 12.2 mg of N′-((1r,4r)-4-morpholinocyclohexyl)-3-phenyl-N⁶-(1-(tetrahydro-2H-pyran-4-yl)-1H-pyrazol-4-yl)-1H-pyrazolo[3,4-d]pyrimidine-4,6-diamine 5 (22 μmol, 61% yield). MS m/z (ESI): 544 [M+1]. ¹H NMR (400 MHz, Methanol-d₄) δ 8.06 (s, 1H), 7.68-7.66 (m, 3H), 7.60-7.53 (m, 3H), 4.61 (s, 11H), 4.42-4.35 (m, 1H), 4.11-4.03 (m, 3H), 3.72 (t, 4H), 3.64-3.57 (m, 2H), 2.63 (m, 4H), 2.30-2.25 (m, 3H), 2.22-2.01 (m, 7H), 1.48-1.13 (m, 5H).

### Example 6: M-(3-methylisothiazol-5-yl)-N′-((1r,4r)-4-morpholinocyclohexyl)-1H-pyrazolo[3,4-d]pyrimidine-4,6-diamine 6

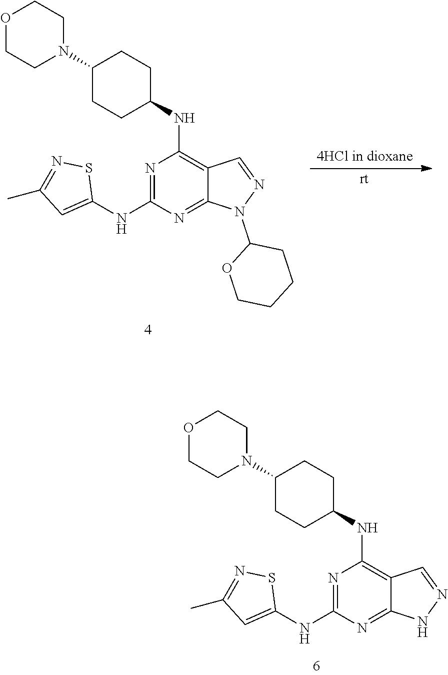 [SMILES: Cc1cc(Nc2nc(N[C@H]3CC[C@H](N4CCOCC4)CC3)c3cnn(C4CCCCO4)c3n2)sn1 ; Cc1cc(Nc2nc(N[C@H]3CC[C@H](N4CCOCC4)CC3)c3cn[nH]c3n2)sn1]

### Step 1. Synthesis of N⁶-(3-methylisothiazol-5-yl)-N⁴-((1r,4r)-4-morpholinocyclohexyl)-1H-pyrazolo[3,4-d]pyrimidine-4,6-diamine 6

To a stirred solution of N⁶-(3-methylisothiazol-5-yl)-N⁴-((1r,4r)-4-morpholinocyclohexyl)-1-(tetrahydro-2H-pyran-2-yl)-1H-pyrazolo[3,4-d]pyrimidine-4,6-diamine 4 (80 mg, 0.16 mmol) in dioxane (2 ml, Aldrich) was added 4HCl in dioxane (2 ml, Aldrich) and the resulting mixture was stirred at rt overnight. The solvent was removed and the residue was washed with ether to afford the title compound 6 (65 mg, 90%). MS m/z (ESI): 415 [M+1].

### Example 7: 3-bromo-N⁶-(3-methylisothiazol-5-yl)-N⁴-((1r,4r)-4-morpholinocyclohexyl)-1H-pyrazolo[3,4-d]pyrimidine-4,6-diamine 7

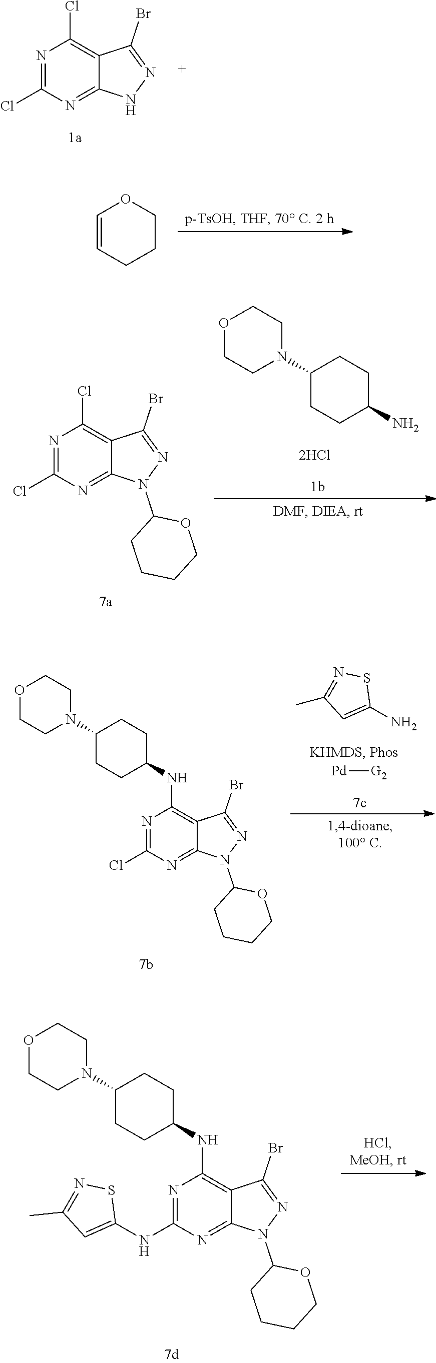

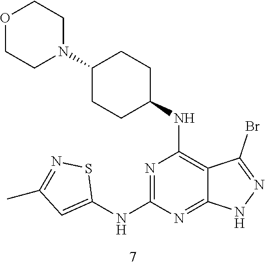 [SMILES: Cc1cc(Nc2nc(N[C@H]3CC[C@H](N4CCOCC4)CC3)c3c(Br)n[nH]c3n2)sn1]

### Step 1. Synthesis of 3-bromo-4,6-dichloro-1-(tetrahydro-2H-pyran-2-yl)-1H-pyrazolo[3,4-d]pyrimidine 7a

To a solution of 3-bromo-4,6-dichloro-1H-pyrazolo[3,4-d]pyrimidine 1a (1 g, 3.73 mmol, Ambeed) in anhydrous THF (16 mL), was added 3,4-dihydro-2H-pyran (1.03 mL, 11.2 mmol, Combi-Blocks) and p-toluenesulfonic acid monohydrate (142.5 mg, 0.75 mmol, Aldrich). The resulting mixture was under reflux for 2 h. The mixture was then concentrated in vacuo, and the residue was triturated with ether (8 mL) at 40° C. for 2 h. The resulting suspension was allowed to cooled to rt. Filtration followed by washing of the residue gave 986 mg of 3-bromo-4,6-dichloro-1-(tetrahydro-2H-pyran-2-yl)-1H-pyrazolo[3,4-d]pyrimidine 7a (2.8 mmol, 75% yield).

### Step 2. Synthesis of 3-bromo-6-chloro-N-((1r,4r)-4-morpholinocyclohexyl)-1-(tetrahydro-2H-pyran-2-yl)-1H-pyrazolo[3,4-d]pyrimidin-4-amine 7b

To a solution of trans-4-morpholinocyclohexan-1-amine dihydrochloride 1b (756.2 mg, 2.94 mol, Combi-Blocks) in anhydrous DMF (10 mL), was added N-ethyl-N-isopropylpropan-2-amine (DIEA, 1.19 g, 9.24 mmol, Aldrich), followed by 3-bromo-4,6-dichloro-1-(tetrahydro-2H-pyran-2-yl)-1H-pyrazolo[3,4-d]pyrimidine 7a (986 mg, 2.8 mmol). The resulting mixture was stirred at rt for 20 h. The mixture was diluted with 20 mL of ethyl acetate and 10 mL of water. The organic layer was separated, and the aqueous layer was extracted with ethyl acetate (10 mL). The combined organic layers were washed with water and brine, dried over sodium sulfate, filtered and concentrated. The crude product 7b was used for next step without further purification.

### Step 3. Synthesis of 3-bromo-N⁶-(3-methylisothiazol-5-yl)-N⁴-((1r,4r)-4-morpholinocyclohexyl)-1-(tetrahydro-2H-pyran-2-yl)-1H-pyrazolo[3,4-d]pyrimidine-4,6-diamine 7d

To a solution of crude product 7b obtained from above reaction (˜2.8 mmol) in anhydrous 1,4-dioxane (15 mL), was added 639.5 mg of 3-methylisothiazol-5-amine 7c (5.6 mmol, Ambeed), X-Phos Pd-G2 catalyst (110 mg, 0.14 mmol, Aldrich) and potassium bis(trimethylsilyl)amide (KHMDS, 1 M THF solution, 5.6 mL, 5.6 mmol, Aldrich). The resulting mixture was bubbled with nitrogen for 2 min before it was sealed and heated at 100° C. for 20 h. After being cooled to rt, the mixture was diluted with 20 mL of ethyl acetate and 10 mL of water. The organic layer was separated, and the aqueous layer was extracted with ethyl acetate (20 mL). The combined organic layers were washed with brine, dried over sodium sulfate, filtered and concentrated. The residue was purified by column chromatography (0%-10% methanol in DCM) to give 141 mg of 3-bromo-N⁶-(3-methylisothiazol-5-yl)-N⁴-((1r,4r)-4-morpholinocyclohexyl)-1-(tetrahydro-2H-pyran-2-yl)-1H-pyrazolo[3,4-d]pyrimidine-4,6-diamine 7d (0.244 mmol, 9% yield for two step).

### Step 4. Synthesis of 3-bromo-N⁶-(3-methylisothiazol-5-yl)-N⁴-((1r,4r)-4-morpholinocyclohexyl)-1H-pyrazolo[3,4-d]pyrimidine-4,6-diamine 7

To a solution of 3-bromo-N⁶-(3-methylisothiazol-5-yl)-N⁴-((1r,4r)-4-morpholinocyclohexyl)-1-(tetrahydro-2H-pyran-2-yl)-1/H-pyrazolo[3,4-d]pyrimidine-4,6-diamine 7d (70 mg, 0.12 mmol) in methanol (1 mL), was added HCl solution (4 M in 1,4-dioxane, 0.3 mL, 1.2 mmol, Aldrich). The resulting mixture was stirred at it for 20 h. The mixture was then concentrated in vacuo, and the residue was diluted with 2 mL ethyl acetate and 1 mL of saturated sodium carbonate solution. The residue was filtered off and was washed with water (2 mL) twice and ethyl acetate (1 mL). The residue was air-dried to give 36.5 mg of 3-bromo-N⁶-(3-methylisothiazol-5-yl)-N′-((1r,4r)-4-morpholinocyclohexyl)-1H-pyrazolo[3,4-d]pyrimidine-4,6-diamine 7 (0.094 mmol, 79% yield). MS m/z (ESI): 493 [M+1]. ¹H NMR (400 MHz, DMSO-d₆) δ 13.34 (s, 1H), 11.03 (s, 1H), 6.82 (m, 1H), 6.41 (m, 1H), 4.32 (m, 1H), 3.59 (m, 5H), 2.27 (m, 5H), 2.13 (m, 3H), 2.00-1.98 (m, 2H), 1.53-1.40 (m, 5H).

### Biological Assays

The present disclosure will be further described with reference to the following test examples, but the examples should not be considered as limiting the scope of the disclosure.

### Test Example 1. IRAK4 Biochemical Assay

### Experimental Procedure:

For the routine compound screening, an IRAK4 kinase assay was performed as follows using Promega ADP-Glo IRAK4 Kinase Enzyme System (Promega, Cat #2198-AD). Purified full length human IRAK4 protein (Signal Chem; Cat #112-10G-125) was diluted to a concentration 6 nM in assay buffer (25 mM MOPS, pH 7.2, 12.5 mM β-glycerol-phosphate, 25 mM MgCl2, 5 mM EGTA, 2 mM EDTA, 0.0025% Brij-35, add 0.25 mM DTT to Kinase Assay Buffer prior to use) containing IRAK4 inhibitor at 2× the final concentration in 0.4% DMSO. The reaction was performed in PerkinElmer Proxiplate-384 plus white plate (Fisher Scientific, Cat #50-905-2761) and started by the addition of an equal volume of the assay buffer containing 4 μM native swine myelin basic protein (MBP) and 500 μM ATP to achieve a final concentration of 3 nM enzyme, 2 μM MBP, 250 μM ATP, 1× compound, and 0.2% DMSO. The kinase reaction was allowed to run for 60 min at room temperature (25-27° C.), then 5 μL of ADP-Glo was added to the reaction in each well followed by 40 minutes-incubation at RT. To detect converted ATP, 10 μL Detection Buffer was added into each well and Incubate at RT in dark for 30 minutes. Finally, luminescence was read using Tecan with 1000 ms of integration time. The luminescent signal positively correlates with ADP amount and kinase activity. Data was analyzed by GraphPad Prism and IC50 was calculated using function log(inhibitor) vs. response—Variable slope (four parameters). Percentage of inhibition was calculated as below.

% inhibition=(1−(Reading(Sample)−Reading(Negative Control))/(Reading(Positive Control)−Reading(Negative Control)))×100%

The 0% inhibition value comes from the Reading of Positive Control wells having 0 nM compound. The 100% inhibition value comes from the Reading of Negative Control wells having no IRAK4 kinase.

Data Analysis:

| | IRAK4 | IRAK4 |
|---|---|---|
| | Biochemical | Biochemical |
| Example | Assay | Assay Max |
| No. | (IC₅₀, nM) | Inhibition (%) |
| 1 | 4.2 | 100 |
| 2 | 8.2 | 100 |
| 3 | 12 | 109 |
| 5 | 16 | 100 |
| 6 | 12 | 100 |

Conclusion: The compounds of the present disclosure have a significant inhibition effect on the enzymatic activity of IRAK4.

### Test Example 2. IRAK4 Human PBMC TNFα Cell Based Assay

### Experimental Procedure:

In this assay, serially diluted compounds were incubated with 1×10⁴ human peripheral blood mononuclear cells (PBMCs) from STEMCELL (Cat #70025.1) per well of 384-well plate (Fisher Scientific; 50-905-2761) and cultured with TexMACS medium (Miltenyi, Cat #130-097-196), stimulated by R848 (0.5 μM) or IL-1β (10 ng/ml). Plates were covered with lids and incubated for 4 h (R848, Fisher Scientific, Cat #NC9801605) or 24 h (IL-1β, Fisher Scientific, Cat #ENRIL1BI) at 37° C. in a humidified tissue-culture incubator. After a brief centrifugation at 1000 rpm for 5 minutes, 5 μl of culture medium was used for the measurement of the TNFα amount using TNFα (human) AlphaLISA Detection Kit (PerkinElmer, Cat #AL208C) according to manufacturer's protocol. A biotinylated anti-TNFα antibody binds to the Streptavidin-coated Donor beads while another anti-TNFα antibody is conjugated to AlphaLISA Acceptor beads. In the presence of the TNFα, the beads come into close proximity. The excitation of the Donor beads causes the release of singlet oxygen molecules that triggers a cascade of energy transfer in the Acceptor beads, resulting in a sharp peak of light emission at 615 nm. AlphaLISA assay plates were read using PHERAstar. Percentage of inhibition was calculated as below:

% inhibition=(1−(Reading(Sample)−Reading(Negative Control))/(Reading(Positive Control)−Reading(Negative Control)))×100%

The 0% inhibition value comes from the Reading of Positive Control wells having 0 nM compound. The 100% inhibition value comes from the Reading of Negative Control wells having no R848 or IL-1β.

### Data Analysis:

| | IRAK4 | IRAK4 |
|---|---|---|
| | PBMC | PBMC |
| | AlphaLISA | AlphaLISA |
| Example | R848 | IL-1β |
| No. | (IC₅₀, nM) | (IC₅₀, nM) |
| 1 | 35 | 16 |
| 2 | 24 | 25 |
| 5 | 63 | 56 |
| 6 | 1.6 | 0.9 |

Conclusion: The compounds of the present disclosure have a significant inhibition effect on the cellular activity of IRAK4.

The foregoing embodiments and examples are provided for illustration only and are not intended to limit the scope of the invention. All patent or non-patent references cited are incorporated herein by reference in their entireties without admission of them as prior art.

What is claimed is:

1. A compound of formula (I) or (I′), or a tautomer, mesomer, racemate, enantiomer, diastereomer, or mixture thereof, or a pharmaceutically acceptable salt thereof: 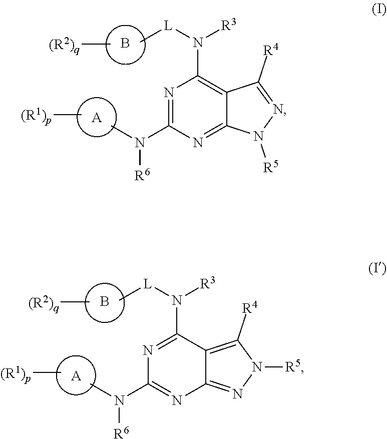
wherein:
ring A is heteroaryl;
ring B is cycloalkyl, heterocyclyl, or heteroaryl, provided that ring B is not piperidinyl, bridged piperidinyl, or cyclopropyl;
L is a covalent bond or alkylene having one CH₂ group optionally replaced with O or NH;
R¹ at each occurrence is identical or different and is independently selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R² at each occurrence is identical or different and is independently selected from halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl, and heteroaryl, wherein the alkyl, cycloalkyl, heterocyclyl, aryl, or heteroaryl is optionally substituted with one or more groups independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R³, R⁶, and R^{d} are each independently selected from hydrogen, alkyl, haloalkyl, hydroxyalkyl, and cycloalkyl, wherein the cycloalkyl is optionally substituted with one or more groups independently selected from halogen, alkyl, haloalkyl, alkoxy, haloalkoxy, hydroxyl, hydroxyalkyl, cyano, amino, and nitro;
R⁴ is selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R⁵ is selected from hydrogen, alkyl, haloalkyl, hydroxyalkyl, —C(═O)R^{c}, —C(═O)OR^{a}, —C(═O)NR^{a}R^{b}, —SO₂R^{a}, —SO₂NR^{a}R^{b}, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R^{a} and R^{b} are identical or different and at each occurrence are independently selected from hydrogen, alkyl, haloalkyl, hydroxyalkyl, cycloalkyl, heterocyclyl, aryl and heteroaryl; or R^{a} and R^{b} together with the nitrogen to which they are attached form a heterocyclyl;
R^{e} and R^{f} are identical or different and at each occurrence are independently selected from hydrogen, alkyl, haloalkyl, hydroxyalkyl, cycloalkyl, heterocyclyl, aryl, and heteroaryl; or R^{e} and R^{f} together with the nitrogen to which they are attached form a heterocyclyl;
R^{c} at each occurrence is independently selected from alkyl, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, and nitro;
p is 0, 1, 2, 3 or 4;
q is 1, 2, 3 or 4;
r is 0, 1, 2 or 3; and
s is 0, 1, 2 or 3.

2. The compound of claim 1, or a tautomer, mesomer, racemate, enantiomer, diastereomer, or mixture thereof, or a pharmaceutically acceptable salt thereof, wherein ring B is C₄-C₆ cycloalkyl.

3. The compound of claim 1, or a tautomer, mesomer, racemate, enantiomer, diastereomer, or mixture thereof, or a pharmaceutically acceptable salt thereof, being a compound of formula (II) or (II′), or a tautomer, mesomer, racemate, enantiomer, diastereomer, or mixture thereof, or a pharmaceutically acceptable salt thereof: 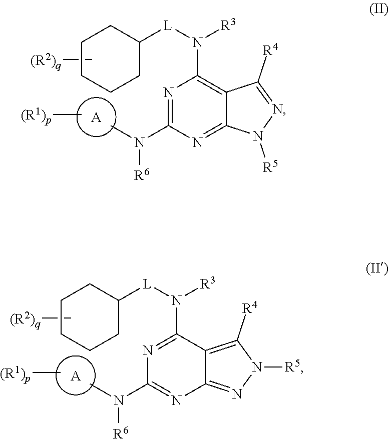
wherein ring A, L, R¹ to R⁶, p and q are each as defined in claim 1.

4. The compound of claim 1, or a tautomer, mesomer, racemate, enantiomer, diastereomer, or mixture thereof, or a pharmaceutically acceptable salt thereof, being a compound of formula (III) or (III′), or a tautomer, mesomer, racemate, enantiomer, diastereomer, or mixture thereof, or a pharmaceutically acceptable salt thereof: 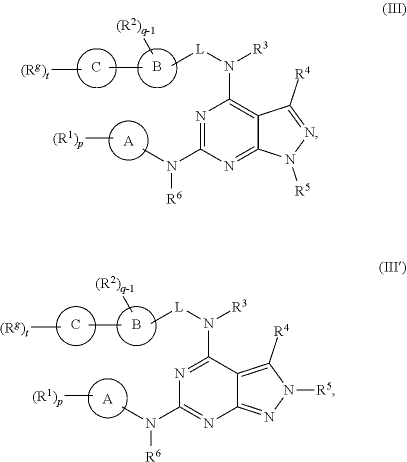
wherein:
ring C is selected from cycloalkyl, heterocyclyl, aryl, and heteroaryl;
R^{g} at each occurrence is identical or different and is independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
t is 0, 1, 2 or 3;
ring A, ring B, L, R¹ to R⁶, R^{f}, R^{e}, s, p and q are each as defined in claim 1.

5. The compound of claim 4, or a tautomer, mesomer, racemate, enantiomer, diastereomer, or mixture thereof, or a pharmaceutically acceptable salt thereof, being a compound of formula (IV), (IV′), (IVa) or (IV′a), or a tautomer, mesomer, racemate, enantiomer, diastereomer, or mixture thereof, or a pharmaceutically acceptable salt thereof: 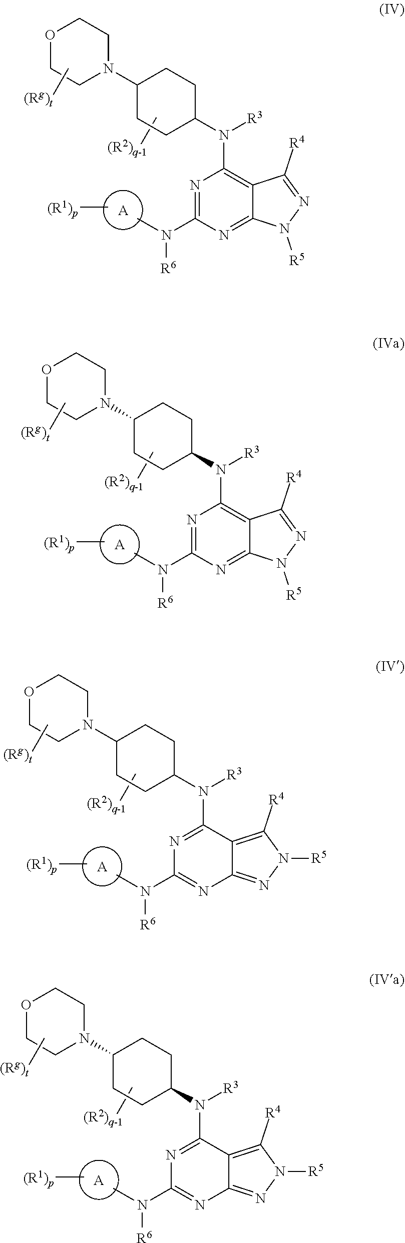
wherein:
ring A, R¹ to R⁶, R^{g}, t, p and q are each as defined in claim 4.

6. The compound of claim 1, or a tautomer, mesomer, racemate, enantiomer, diastereomer, or mixture thereof, or a pharmaceutically acceptable salt thereof, wherein ring A is isothiazolyl or pyrazolyl.

7. The compound of claim 1, or a tautomer, mesomer, racemate, enantiomer, diastereomer, or mixture thereof, or a pharmaceutically acceptable salt thereof, wherein R¹ is C₁₋₆, alkyl or 3-8 membered heterocyclyl.

8. The compound of claim 1, or a tautomer, mesomer, racemate, enantiomer, diastereomer, or mixture thereof, or a pharmaceutically acceptable salt thereof, wherein R² is morpholinyl.

9. The compound of claim 1, or a tautomer, mesomer, racemate, enantiomer, diastereomer, or mixture thereof, or a pharmaceutically acceptable salt thereof, wherein R³ is hydrogen.

10. The compound of claim 1, or a tautomer, mesomer, racemate, enantiomer, diastereomer, or mixture thereof, or a pharmaceutically acceptable salt thereof, wherein R⁴ is selected from hydrogen, halogen, 6-10 membered aryl, and 5-10 membered heteroaryl.

11. The compound of claim 1, or a tautomer, mesomer, racemate, enantiomer, diastereomer, or mixture thereof, or a pharmaceutically acceptable salt thereof, wherein each R⁵ is hydrogen or 3-8 membered heterocyclyl.

12. The compound of claim 1, or a tautomer, mesomer, racemate, enantiomer, diastereomer, or mixture thereof, or a pharmaceutically acceptable salt thereof, wherein each L is a covalent bond.

13. The compound of claim 1, or a tautomer, mesomer, racemate, enantiomer, diastereomer, or mixture thereof, or a pharmaceutically acceptable salt thereof, wherein R^{g} is hydrogen.

14. The compound of claim 1, or a tautomer, mesomer, racemate, enantiomer, diastereomer, or mixture thereof, or a pharmaceutically acceptable salt thereof, wherein the compound is selected from:

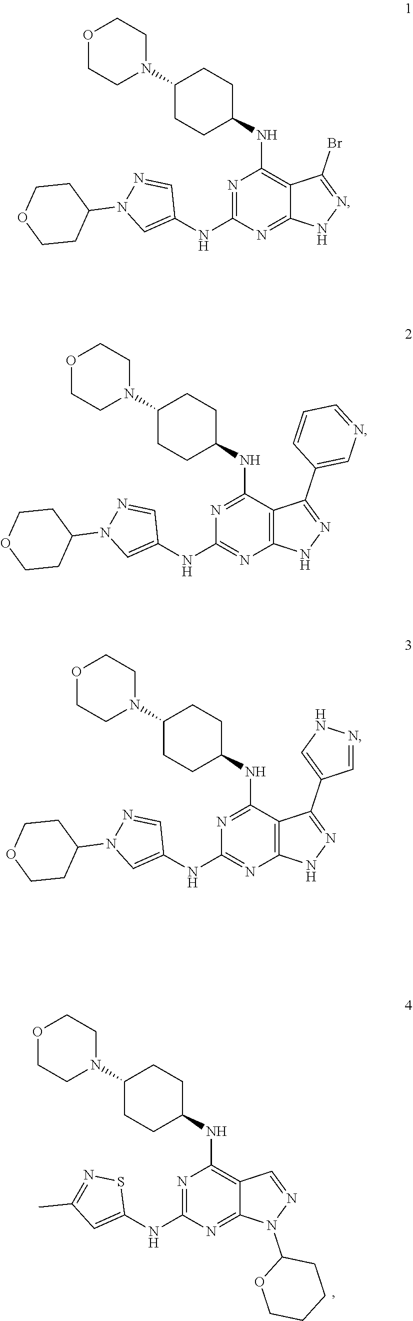 [SMILES: Brc1n[nH]c2nc(Nc3cnn(C4CCOCC4)c3)nc(N[C@H]3CC[C@H](N4CCOCC4)CC3)c12 ; c1cncc(-c2n[nH]c3nc(Nc4cnn(C5CCOCC5)c4)nc(N[C@H]4CC[C@H](N5CCOCC5)CC4)c23)c1 ; c1nn(C2CCOCC2)cc1Nc1nc(N[C@H]2CC[C@H](N3CCOCC3)CC2)c2c(-c3cn[nH]c3)n[nH]c2n1 ; Cc1cc(Nc2nc(N[C@H]3CC[C@H](N4CCOCC4)CC3)c3cnn(C4CCCCO4)c3n2)sn1]

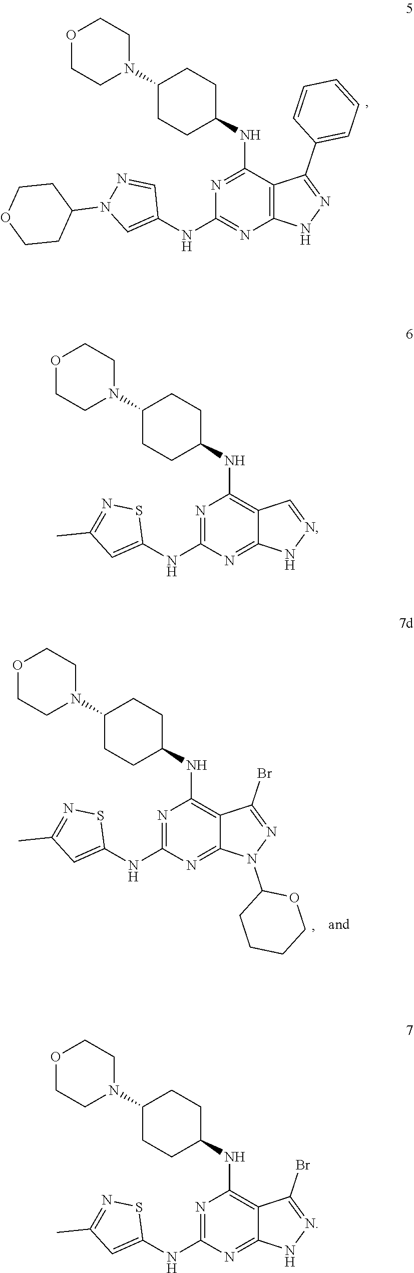 [SMILES: c1ccc(-c2n[nH]c3nc(Nc4cnn(C5CCOCC5)c4)nc(N[C@H]4CC[C@H](N5CCOCC5)CC4)c23)cc1 ; Cc1cc(Nc2nc(N[C@H]3CC[C@H](N4CCOCC4)CC3)c3cn[nH]c3n2)sn1 ; Cc1cc(Nc2nc(N[C@H]3CC[C@H](N4CCOCC4)CC3)c3c(Br)nn(C4CCCCO4)c3n2)sn1 ; Cc1cc(Nc2nc(N[C@H]3CC[C@H](N4CCOCC4)CC3)c3c(Br)n[nH]c3n2)sn1]

15. A compound of formula (IIIA2) or (III′A2), or a tautomer, mesomer, racemate, enantiomer, diastereomer, or mixture thereof, or a pharmaceutically acceptable salt thereof: 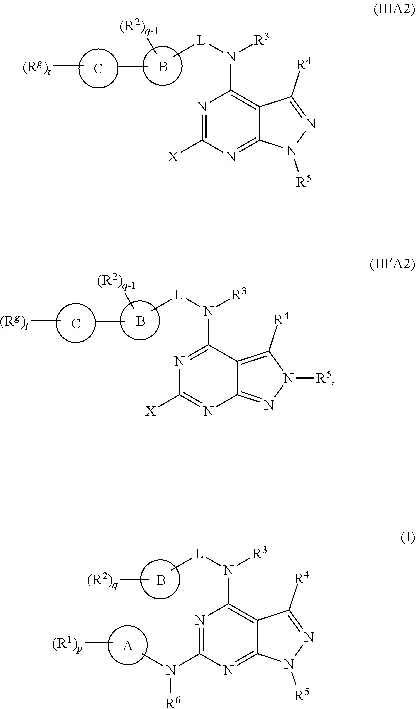
wherein:
X is halogen;
ring B is heterocyclyl or C₄₋₆ cycloalkyl;
ring C is selected from heterocyclyl, aryl, heteroaryl, and C₄₋₆ cycloalkyl;
R^{g} at each occurrence is identical or different and is independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
L is a covalent bond or alkylene having one CH₂ group optionally replaced with O or NH;
R² at each occurrence is identical or different and is independently selected from halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl, and heteroaryl, wherein the alkyl, cycloalkyl, heterocyclyl, aryl, or heteroaryl is optionally substituted with one or more groups independently selected from hydrogen, halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R³, R⁶, and R^{d} are each independently selected from hydrogen, alkyl, haloalkyl, hydroxyalkyl, and cycloalkyl, wherein the cycloalkyl is optionally substituted with one or more groups independently selected from halogen, alkyl, haloalkyl, alkoxy, haloalkoxy, hydroxyl, hydroxyalkyl, cyano, amino, and nitro;
R⁴ is selected from hydrogen, halogen, alkyl, alkoxy, haloalkyl, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R⁵ is selected from hydrogen, alkyl, haloalkyl, hydroxyalkyl, —C(═O)R^{c}, —C(═O)OR^{a}, —C(═O)NR^{a}R^{b}, —SO₂R^{a}, —SO₂NR^{a}R^{b}, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl, and heteroaryl;
R^{a} and R^{b} are identical or different and at each occurrence are independently selected from hydrogen, alkyl, haloalkyl, hydroxyalkyl, cycloalkyl, heterocyclyl, aryl and heteroaryl; or R^{a} and R^{b} together with the nitrogen to which they are attached form a heterocyclyl;
R^{e} and R^{f} are identical or different and at each occurrence are independently selected from hydrogen, alkyl, haloalkyl, hydroxyalkyl, cycloalkyl, heterocyclyl, aryl, and heteroaryl; or R^{e} and R^{f} together with the nitrogen to which they are attached form a heterocyclyl;
R^{c} at each occurrence is independently selected from alkyl, cycloalkyl, heterocyclyl, aryl, and heteroaryl, wherein the alkyl, cycloalkyl, heterocyclyl, aryl, or heteroaryl is optionally substituted with one or more groups independently selected from halogen, alkyl, haloalkyl, alkoxy, haloalkoxy, hydroxyl, hydroxyalkyl, cyano, amino, and nitro;
p is 0, 1, 2, 3 or 4;
q is 1, 2, 3 or 4;
r is 0, 1, 2 or 3;
s is 0, 1, 2 or 3; and
t is 0, 1, 2 or 3.

16. The compound of claim 15, or a tautomer, mesomer, racemate, enantiomer, diastereomer, or mixture thereof, or a pharmaceutically acceptable salt thereof, wherein the compound is selected from:

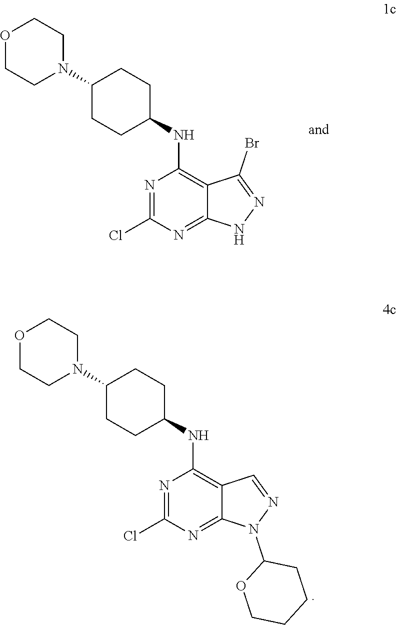 [SMILES: Clc1nc(N[C@H]2CC[C@H](N3CCOCC3)CC2)c2c(Br)n[nH]c2n1 ; Clc1nc(N[C@H]2CC[C@H](N3CCOCC3)CC2)c2cnn(C3CCCCO3)c2n1]

17. A process of preparing the compound of formula (I) or (I′) according to claim 1, or a tautomer, mesomer, racemate, enantiomer, diastereomer, or mixture thereof, or a pharmaceutically acceptable salt thereof, comprising a step of: 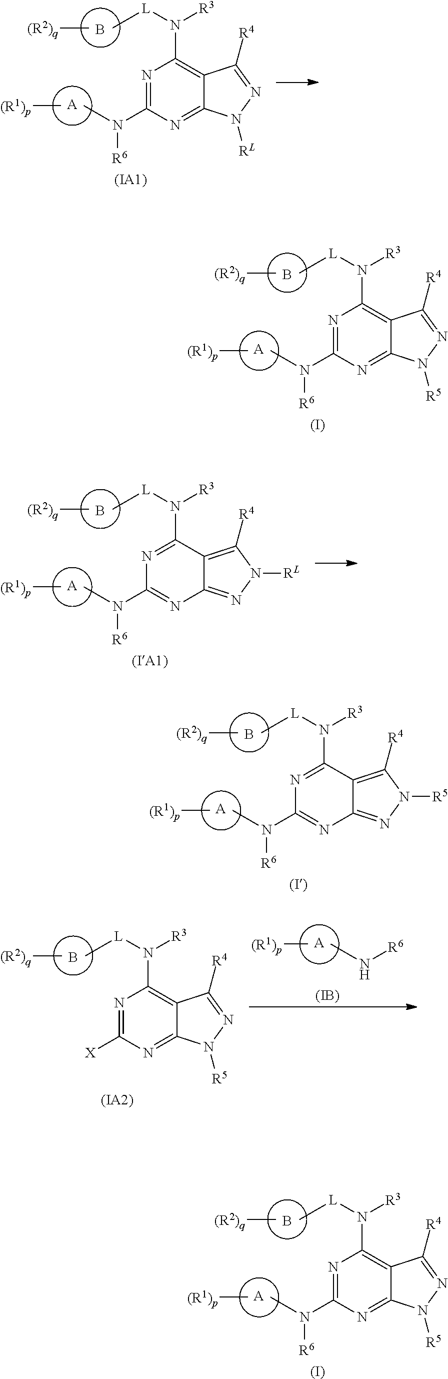 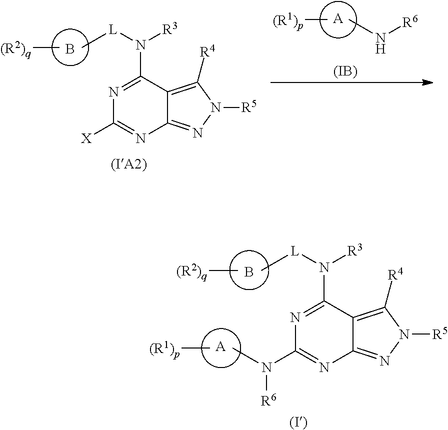
removing the group R^{L} from the compound of formula (IA1) or (I′A1) or reacting a compound of formula (IA2) or (I′A2) with a compound of formula (IB) or a salt thereof to obtain the compound of formula (I) or (I′), wherein:
R^{L} is a protecting group, preferably tetrahydropyranyl;
R⁵ is H; and
ring A, ring B, L, R¹ to R⁴, R⁶, p and q are each as defined in claim 1.

18. A pharmaceutical composition, comprising a compound of claim 1, or a tautomer, mesomer, racemate, enantiomer, diastereomer, or mixture thereof, or a pharmaceutically acceptable salt thereof, and a pharmaceutically acceptable carrier.

19. A method of preventing and/or treating an IRAK-mediated disorder, disease, or condition, comprising administering to a subject in need thereof a therapeutically effective amount of a compound of claim 1, or a tautomer, mesomer, racemate, enantiomer, diastereomer, or mixture thereof, or a pharmaceutically acceptable salt thereof, or the pharmaceutical composition thereof; wherein the IRAK-mediated disorder, disease or condition is selected from an autoimmune disease, an inflammatory disorder, a cancer, a viral disease, a neurodegenerative disorder, a hereditary disorder, a hormone-related disease, a metabolic disorder, conditions associated with organ transplantation, immunodeficiency disorders, a destructive bone disorder, a proliferative disorder, an infectious disease, pathologic immune conditions involving T cell activation, a cardiovascular disorder, and a CNS disorder.

20. The method of claim 19, wherein the cancer or proliferative disorder is selected a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, large cell carcinoma, nonsmall-cell lung carcinoma, lymphomas, Hodgkins and Non-Hodgkins, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABCDLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulmemia (WM), splenic marginal zone lymphoma, multiple myeloma, plasmacytoma, and intravascular large B-cell lymphoma); and wherein: the MyD88 driven disorder is selected from ABC DLBCL, Waldenstrom's macroglobulmemia, Hodgkin's lymphoma, primary cutaneous T-cell lymphoma and chronic lymphocytic leukemia; the IL-1 driven disorder is Smoldering of indolent multiple myeloma; the neurodegenerative disease is selected from Alzheimer's disease, Parkinson's disease, Huntington's disease, glutamate neurotoxicity, hypoxia, epilepsy, treatment of diabetes, metabolic syndrome, obesity; the inflammatory disorder is selected from conditions of the eye such as ocular allergy, conjunctivitis, keratoconjunctivitis sicca, and vernal conjunctivitis; and inflammatory disease in which autoimmune reactions are implicated or having an autoimmune component or etiology, including autoimmune hematological disorders, systemic lupus erythematosus, rheumatoid arthritis, polychondritis, scleroderma, Wegener granulomatosis, dermatomyositis, chronic active hepatitis, myasthenia gravis, autoimmune inflammatory bowel disease, irritable bowel syndrome, kidney disease, glomerular disease, alcoholic liver disease, multiple sclerosis, endocrine ophthalmopathy, Grave's disease, chronic hypersensitivity pneumonitis, multiple sclerosis, primary biliary cirrhosis, uveitis (anterior and posterior), Sjogren's syndrome, systemic juvenile idiopathic arthritis, nephritis, chronic granulomatous disease, obesity, fetal growth retardation, hypercholesterolemia, heart disease, chronic heart failure, mesothelioma, Behcet's disease, pancreatitis, asthma, acute lung injury, acute respiratory distress syndrome, eosinophilia, hypersensitivities, anaphylaxis, nasal sinusitis, ocular allergy, silica induced diseases, COPD, pulmonary disease, cystic fibrosis, cataracts, muscle inflammation in conjunction with systemic sclerosis, inclusion body myositis, myasthenia gravis, thyroiditis, Addison's disease, lichen planus, Type 1 diabetes, or Type 2 diabetes, asthma, allergy, blepharitis, bronchiolitis, bronchitis, colitis, conjunctivitis, cystitis, dacryoadenitis, dermatitis, endometritis, enteritis, enterocolitis, epicondylitis, epididymitis, fasciitis, fibrositis, gastritis, gastroenteritis, Henoch-Schonlein purpura, hepatitis, hidradenitis suppurativa, meningitis, myositis, nephritis, pancreatitis, pneumonia, synovitis, ulcerative colitis, uveitis, vaginitis, alopecia areata, erythema multiforma, dermatitis, scleroderma, vitiligo, hypersensitivity angiitis, urticaria, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.
